(19) United States
(12) Patent Application Publication
(10) Pub. No.: US 2008/0275035 A1
(43) Pub. Date: Nov. 6, 2008

(54) Nitroimidazole Compounds

(76) Inventors: Jan Jiricek, Singapore (SG); Sejal Patel, Singapore (SG); Thomas Hugo Keller, Singapore (SG); Clifton E. Barry, Germantown, MD (US); Cynthia S. Dowd, Washington, DC (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/097,976

(22) PCT Filed: Dec. 22, 2006
(86) PCT No.: PCT/US06/48763

§ 371 Date: Jun. 18, 2008

Related U.S. Application Data
(60) Provisional application No. 60/753,763, filed on Dec. 23, 2005.
(60) Provisional application No. 60/753,781, filed on Dec. 23, 2005.

(51) Int. Cl. A61K 31/5365; C07D 233/88; C07D 498/04; A61P 33/02; A61K 31/4164

(52) U.S. Cl. 514/230.5; 548/330.1; 544/91; 514/398

(57) ABSTRACT

The present invention relates to certain nitroimidazole compounds, which have interesting pharmaceutical properties. In particular, the compounds are useful in the treatment and/or prevention of infections such as those caused by Mycobacterium tuberculosis, Trypanosoma cruzi or Leishmania donovani. The invention also relates to pharmaceutical compositions containing the compounds, as well as processes for their preparation.

### TECHNICAL FIELD

The present invention relates to nitroimidazole compounds, to processes for their production, their use as pharmaceuticals and pharmaceutical compositions comprising them.

### BACKGROUND

Tuberculosis (TB), one of the oldest diseases known to humankind, is caused by the bacterium Mycobacterium tuberculosis (MTB). The disease is contagious and, like the common cold, can be easily spread through air by coughing and sneezing. Currently, MTB infects one-third of the world population and is the second leading cause of adult mortality by an infectious disease after AIDS, with one TB death every 15 seconds. For the last two decades, there has been a resurgence of TB cases, particularly in areas like South East Asia and sub-Saharan Africa.

The first effective anti-TB drug, streptomycin, was introduced in 1946. However, the monotherapy became rapidly ineffective because of the development of bacterial resistance. As more anti-mycobacterials were discovered, combination therapies with two or more drugs were possible to suppress the emergence of resistance. The last new anti-TB drug, rifampicin, was introduced in the 1960s. For the last thirty years, no TB drug with a new mode of action has been introduced. Current therapy for TB is effective but involves multiple drugs. This involves the initial treatment with rifampicin, isoniazid, pyrazinamide and ethambutol for two months, followed by continuation therapy with rifampicin and isoniazid for another 4 months. The major shortcoming of this regimen is the long treatment time, which makes patient compliance and proper implementation a challenge. More than two-thirds of the TB patients do not receive full and proper TB treatment, which results in a high relapse rate and emergence of drug resistance. Currently, about 4% of the TB cases worldwide are multiple-drug resistant (MDR) i.e. resistant to both isoniazid and rifampicin. MDR-TB is difficult to cure, with treatment time up to 2 years and a high failure rate. Novel TB drugs are urgently needed to shorten treatment time and to treat multi-drug resistant TB in a more effective way.

Leishmaniasis is caused by one of more than 20 varieties of parasitic protozoa that belong to the genus Leishmania, and is transmitted by the bite of female sandflies. Leishmaniasis is endemic in about 88 countries, including many tropical and sub-tropical areas.

There are four main forms of leishmaniasis. Visceral leishmaniasis, also called kala-azar, is the most serious form and is caused by the parasite Leishmania donovani. Patients who develop visceral leishmaniasis can die within months unless they receive treatment. The two main therapies for visceral leishmaniasis are the antimony derivatives sodium stibogluconate (Pentostam®) and meglumine antimoniate (Glucantim®). Sodium stibogluconate has been used for about 70 years and resistance to this drug is a growing problem. In addition, the treatment is relatively long and painful, and can cause undesirable side effects.

Chagas disease (also called American trypanosomiasis) is another human parasitic disease that is endemic amongst poor populations on the American continent. The disease is caused by the protozoan parasite Trypanosoma cruzi, which is transmitted to humans by blood-sucking insects. The human disease occurs in two stages: the acute stage, which occurs shortly after the infection, and the chronic stage, which can develop over many years. Chronic infections result in various neurological disorders, including dementia, damage to the heart muscle and sometimes dilation of the digestive tract, as well as weight loss. Untreated, the chronic disease is often fatal.

The drugs currently available for treating Chagas disease are Nifurtimox and Benznidazole. However, problems with these current therapies include their adverse side effects, the length of treatment, and the requirement for medical supervision during treatment. Furthermore, treatment is really only effective when given during the acute stage of the disease. Resistance to the two frontline drugs has already arisen. The antifungal agent Amphotericin b has been proposed as a second-line drug, but this drug is costly and relatively toxic.

There is therefore also a need for new drugs that improve current treatments of Leishmaniasis and Chagas disease.

WO97/01562 discloses a number of nitroimidazole compounds, inter alia PA-824, which is useful for treating TB. However, PA-824 has an expensive route of synthesis, a complicated tablet formulation (due to its low solubility) and there is a need for further improvement in efficacy.

It is an object of the present invention to provide improved compounds which are suitable for pharmaceutical uses, preferably having a simple route of synthesis and improved properties with respect to efficacy, solubility and stability.

It has surprisingly been found that nitroimidazole compounds of the present invention have advantageous properties for pharmaceutical use.

### SUMMARY OF INVENTION

In a first aspect the invention provides a compound of formula (I), or a pharmaceutically acceptable salt, ester or prodrug thereof:

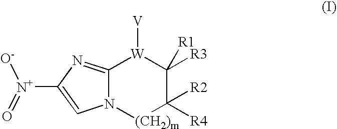

wherein:
(a) m is 0;
   W is O and V is absent;
   one of R1 and R3 is haloaryl and the other is H; and
   R2 and R4 are both H;
or:
(b) m is 1;
   W is N and V is an alkylaryl group, optionally substituted with one or more alkoxy substituents;
   R1 and R3 are both H; and
   one of R2 and R4 is alkoxy and the other is H;
or:
(c) m is1;
   W is O and V is absent;
   one of R1 and R3 is alkyl or aryl, and the other is H; and
   R2 and R4 are both H;
or:
(d) m is 1;
   W is O and V is absent;
   one of R2 and R4 is -L(B)ₙ-(Z)ₚ, -(L-B)_{q}-(Z)ₚ or —Y-(B)_{q}-Z, and the other is H;
   and
   R1 and R3 are both H;
   wherein L is an atom group having of the formula —O—Re where R5 is a lower alkylene, —C(O)—, lower alkylene-C(O)—, —C(O)-lower alkylene, lower alkylene-C(O)—NH—, lower alkylene-NH—; B is a cycloalkyl, heterocyclic, aryl or heteroaryl ring which is optionally further substituted with one or more substituents; n is 1 or 2; and Z is halogen, lower alkyl substituted with at least one halogen, lower alkoxy substituted with at least one halogen or lower thioalkyl substituted with at least one halogen;
   and Y is —NHC(O)—;
   n is 1 or 2; p is 0, 1 or 2; and q is 1 or 2;
   provided that if R2 or R4 is -L-(B)ₙ-(Z)ₚ wherein n is 1, B is phenyl and L is —O—CH₂—, then p is not 0;
   and provided that if R2 or R4 is -(L-B)ₙ-(Z)ₚ wherein q is 2, both B groups are phenyl and L is —O—CH₂—, then p is not 0;
   and provided that if R2 or R4 is -L-(B)ₙ-(Z)ₚ wherein n is 1, B is phenyl and L is —O—CH₂—, then Z is not 4-trifluoromethoxy, 4-fluoro, 4-trifluoroethoxy, 4-pentafluoropropoxy, 4-tetrafluoropropoxy, 4-trifluoromethyl, 2,4-difluoromethyl or 2,4-difluoro.

Preferably the compound is a compound of formula (I) wherein (d) above applies.

Where the compound is a compound of formula (I) wherein (a) above applies, it is preferred that one of R1 and R3 is 4-fluorophenyl.

Where the compound is a compound of formula (I) wherein (b) above applies, it is preferred that V is a benzyl group optionally substituted with one or more methoxy groups. It is further preferred that one of R2 and R4 is a methoxy group.

Where the compound is a compound of formula (I) wherein (c) above applies, it is preferred that one of R1 and R3 is an ethyl, pentyl or phenyl group.

Where the compound is a compound of formula (I) wherein (d) above applies, and wherein one of R2 and R4 is —Y-(B)_{q}-Z it is preferred that B is a piperidine, pyrimidine or phenyl group. It is also preferred that p is 1.

Where the compound is a compound of formula (I) wherein (d) above applies, and wherein one of R2 and R4 is -(L-B)_{q}, (Z)ₚ it is preferred that B is phenyl or cyclohexyl. It is further preferred that L is —O-lower alkylene, more preferably —O—CH₂—.

Where the compound is a compound of formula (I) wherein (d) above applies, and wherein one of R2 and R4 is -L-(B)ₙ-(Z)ₚ it is preferred that B is —OCH₂C(O)—, —OCH₂C(O)NH—, —OCH₂C(O)N—, —OCH₂C(O)NHCH₂—, —OCH₂— or —OCH₂CH₂—; More preferably L is —OCH₂C(O)—. It is further preferred that B is a 4 to 12, preferably 5 or 6 membered cycloalkyl, heterocyclic, aryl or heteroaryl ring. The ring can optionally be further substituted with one or more substituents, preferably selected from the group consisting of lower alkyl, halogen, hydroxy, amino or lower alkoxy. In a preferred embodiment, B is a cyclic ring selected from the group consisting of cyclopentyl, cyclohexyl, phenyl, morpholinyl, piperazinyl, piperidinyl, pyridyl, pyrrolidinyl, pyrazinyl, pyrimidinyl, purinyl, pyranyl, benzimidazolyl, benzoxazolyl, benzthiazolyl, naphthyl, indolyl, indolinyl, quinolinyl, isoquinolinyl, 1,2,3,4-tetrahydroquinolinyl, thiazolyl, imidazolyl, benzotriazolyl, indanyl, oxadiazolyl, pyrazolyl, triazolyl, or tetrazolyl. In a still more preferred embodiment, B is a cyclic ring selected from the group consisting of piperazinyl, phenyl, pyridyl, benzimidazolyl, benzthiazolyl, benzoxazolyl, thiazolyl. Preferably Z is halogen, lower alkyl substituted with at least one halogen or lower alkoxy substituted with at least one halogen, such as e.g. halomethyl, dihalomethyl, trihalomethyl, pentahaloethyl, halomethoxy, dihalomethoxy, trihalomethoxy, pentahaloethyl or pentahaloethoxy.

Where the compound is a compound of formula (I) wherein (d) above applies, and wherein one of R2 and R4 is -L-(B)ₙ-(Z)ₚ it is preferred that B is a piperazine, pyridine, phenyl or benzimidazole group, or an oxazole or thiazole group, optionally fused to a phenyl ring. It is also preferred that p is 1.

Where the compound is a compound of formula (I) wherein (d) above applies, it is preferred that Z is F, Br, trifluoromethyl, trifluoromethoxy or —SCF₃.

Where the compound is a compound of formula (I) wherein (d) above applies, it is preferred that p is 1.

Preferably the compound is a compound of formula (II), or a pharmaceutically acceptable salt, ester or prodrug thereof:

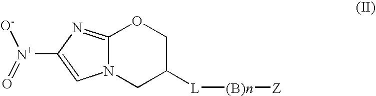

wherein:

L is an atom group having of the formula —OR5 wherein R5 is a lower alkylene, —C(O)—, lower alkylene-C(O)—, —C(O)-lower alkylene, lower alkylene-C(O)—NH—, lower alkylene-NH—; In a preferred embodiment L is selected from the group consisting of —OCH₂C(O)—, —OCH₂C(O)NH—, —OCH₂C(O)N—, —OCH₂C(O)NHCH₂—, —OCH₂— and —OCH₂CH₂—;

B is a 4 to 12, preferably 5 or 6 membered cycloalkyl, heterocyclic, aryl or heteroaryl ring. The ring can optionally be further substituted with one or more substituents, preferably selected from the group consisting of lower alkyl, halogen, hydroxy, amino or lower alkoxy. In a preferred embodiment, B is a cyclic ring selected from the group consisting of cyclopentyl, cyclohexyl, phenyl, morpholinyl, piperazinyl, piperidinyl, pyridyl, pyrrolidinyl, pyrazinyl, pyrimidinyl, purinyl, pyranyl, benzimidazolyl, benzoxazolyl, benzthiazolyl, naphthyl, indolyl, indolinyl, quinolinyl, isoquinolinyl, 1,2,3,4-tetrahydroquinolinyl, thiazolyl, imidazolyl, benzotriazolyl, indanyl, oxadiazolyl, pyrazolyl, triazolyl, or tetrazolyl. In a preferred embodiment, B is a cyclic ring selected from the group consisting of piperazinyl, phenyl, pyridyl, benzimidazolyl, benzthiazolyl, benzoxazolyl, thiazolyl;

Z is halogen, lower alkyl substituted with at least one halogen or lower alkoxy substituted with at least one halogen, such as e.g. halomethyl, dihalomethyl, trihalomethyl, pentahaloethyl, halomethoxy, dihalomethoxy, trihalomethoxy, pentahaloethyl or pentahaloethoxy. Preferably the halogen is fluoro or chloro, fluoro being the most preferred halogen. Z may be attached at any position of the ring structures of B, but is preferably attached at the meta position, more preferably at the para position of the ring structure. In case n=2, Z is preferably attached at the outer cyclic ring, i.e. the cyclic ring which is not directly attached to L, n is 1 or 2, preferably 2;

provided that if n is 1 then B is not phenyl or if n is 1 and B is phenyl then L is —OCH₂C(O)NH— or —OCH₂C(O)NHCH₂—.

In particular, the present invention encompasses 6-substituted-2-nitro-6,7-dihydro-5H-imidazo[2,1-b][1,3]oxazine of formula (I), and 6-substituted-2-nitro-6,7-dihydro-5H-imidazo[2,1-b][1,3]oxazine of formula (II).

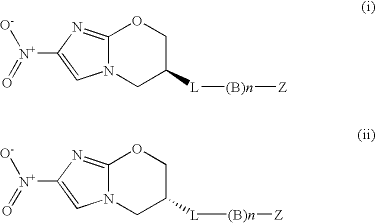

In a preferred embodiment, the present invention relates to a compound, or a pharmaceutically acceptable salt, ester or prodrug thereof, of the formula:

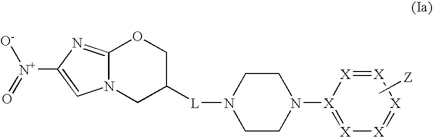

wherein L and Z are as defined above for the compound of formula (II) and wherein X is independently C or N and wherein 0, 1, 2, 3 or 4× are N, provided that the ring structure is chemically stable and wherein each ring can independently be further substituted by 1, 2, 3 or more substituents, e.g. selected from the group consisting of lower alkyl, halogen, hydroxy, amino or lower alkoxy. In a preferred embodiment, not two adjacent X are N, the X attached to N of the other cyclic structure is C, L is selected from the group consisting of —OCH₂C(O)—, —OCH₂C(O)NHCH₂—, —OCH₂—, —OCH₂CH₂— and Z is selected from the group consisting of —F, —CF3, or —OCF₃. Preferably, Z is in the 3-position, more preferably in the 4-position.

In another preferred embodiment, the present invention relates to a compound, or a pharmaceutically acceptable salt, ester or prodrug thereof, of the formula:

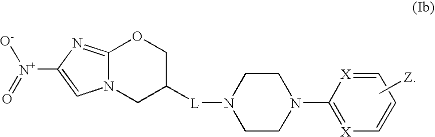

wherein L and Z are as defined above for the compound of formula (II) and wherein X is independently C or N and wherein each ring can independently be further substituted by 1, 2, 3 or more substituents, e.g. selected from the group consisting of lower alkyl, halogen, hydroxy, amino or lower alkoxy. In a preferred embodiment, L is selected from the group consisting of —OCH₂C(O)—, —OCH₂C(O)NHCH₂—, —OCH₂—, —OCH₂CH₁— and Z is selected from the group consisting of —F, —CF₃, or —OCF₃. Preferably, Z is in the 3-position, more preferably in the 4-position.

In another preferred embodiment, the present invention relates to a compound, or a pharmaceutically acceptable salt, ester or prodrug thereof, of the formula:

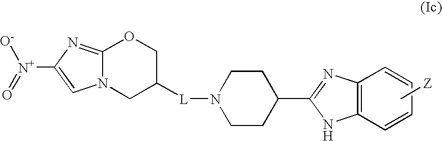

wherein L and Z are as defined above for the compound of formula (II) and wherein each ring can independently be further substituted by 1, 2, 3 or more substituents, e.g. selected from the group consisting of lower alkyl, halogen, hydroxy, amino or lower alkoxy. In a preferred embodiment, L is selected from the group consisting of —OCH₂C(O)—, —OCH₂C(O)NHCH₂—, —OCH₂CH₂— and —OCH₂— and Z is selected from the group consisting of —F, —CF₃, or —OCF₃. Preferably, Z is in position 4 or 7, more preferably in position 5 and 6.

In another preferred embodiment, the present invention relates to a compound, or a pharmaceutically acceptable salt, ester or prodrug thereof, of the formula:

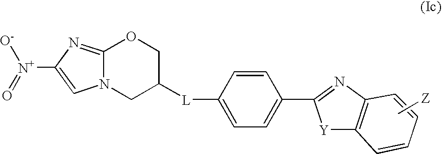

wherein L and Z are as defined above for the compound of formula (II) and wherein Y is O or N and wherein each ring can independently be further substituted by 1, 2, 3 or more substituents, e.g. selected from the group consisting of lower alkyl, halogen, hydroxy, amino or lower alkoxy. In a preferred embodiment, L is selected from the group consisting of —OCH₂C(O)—, —OCH₂C(O)NH—, —OCH₂CO(O)NHCH₂—, —OCH₂CH₂— and —OCH₂— and Z is selected from the group consisting of —F, —CF₃, or —OCF₃. Preferably, Z is in position 4 or 7, more preferably in position 5 and 6.

In another preferred embodiment, the present invention relates to a compound, or a pharmaceutically acceptable salt, ester or prodrug thereof, of the formula:

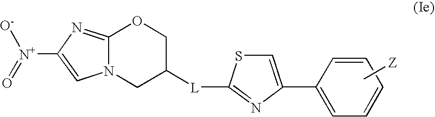

wherein L and Z are as defined above for the compound of formula (II) and wherein each ring can independently be further substituted by 1, 2, 3 or more substituents, e.g. selected from the group consisting of lower alkyl, halogen, hydroxy, amino or lower alkoxy. In a preferred embodiment, L is selected from the group consisting of —OCH₂C(O)—, —OCH₂C(O)NH—, —OCH₂C(O)NHCH₂—, —OCH₂CH₂— and —OCH₂— and Z is selected from the group consisting of —F, —CF₃, or —OCF₃. Preferably, Z is in the 3-position, more preferably in the 4-position.

In another preferred embodiment, the present invention relates to a compound, or a pharmaceutically acceptable salt, ester or prodrug thereof, of the formula:

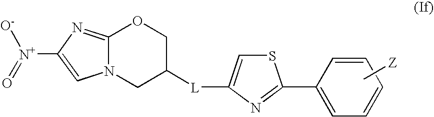

wherein L and Z are as defined above for the compound of formula (II) and wherein each ring can independently be further substituted by 1, 2, 3 or more substituents, e.g. selected from the group consisting of lower alkyl, halogen, hydroxy, amino or lower alkoxy. In a preferred embodiment, L is selected from the group consisting of —OCH₂C(O)—, —OCH₂C(O)NH—, —OCH₂C(O)NHCH₂—, —OCH₂CH₂— and —OCH₂— and Z is selected from the group consisting of —F, —CF₃, or —OCF₃. Preferably, Z is in the 3-position, more preferably in the 4-position.

In another aspect, the present invention relates to a compound, or a pharmaceutically acceptable salt, ester or prodrug thereof, of the formula:

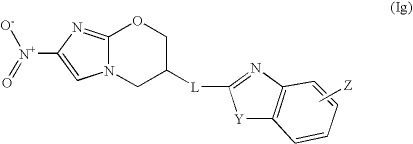

wherein L and Z are as defined above for the compound of formula (II) and wherein Y is S or N and wherein each ring can independently be further substituted by 1, 2, 3 or more substituents, e.g. selected from the group consisting of lower alkyl, halogen, hydroxy, amino or lower alkoxy. In a preferred embodiment, L is selected from the group consisting of —OCH₂C(O)—, —OCH₂C(O)NH—, —OCH₂C(O)NHCH₂—, —OCH₂CH₂— and —OCH₂— and Z is selected from the group consisting of —F—CF₃, or —OCF₃. Preferably, Z is in position 4 or 7, more preferably in position 5 and 6.

In another aspect, the present invention relates to a compound, or a pharmaceutically acceptable salt, ester or prodrug thereof, of the formula:

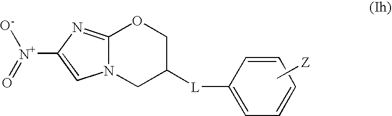

wherein L and Z are as defined above for the compound of formula (II) and wherein each ring can independently be further substituted by 1, 2, 3 or more substituents, e.g. selected from the group consisting of lower alkyl, halogen, hydroxy, amino or lower alkoxy. In a preferred embodiment, L is selected from the group consisting of —OCH₂C(O)—, —OCH₂C(O)NH—, —OCH₂C(O)NHCH₂— and —OCH₂CH₂— and Z is selected from the group consisting of —F, —CF₃, or —OCF₃. Preferably, Z is in the 3-position, more preferably in the 4-position.

In another aspect, the present invention relates to a compound, or a pharmaceutically acceptable salt, ester or prodrug thereof, of the formula:

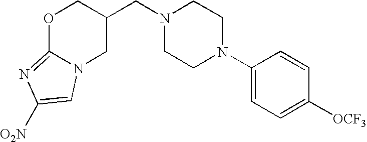

It is preferred that any aryl group in the compound of formula (I) is a phenyl group. The aryl group may be optionally substituted with one or more substituents, preferably selected from lower haloalkyl (more preferably trifluoromethyl), lower haloalkoxy (preferably trifluoromethoxy) or halo (preferably Br, Cl or F, most preferably F).

In another aspect, the present invention relates to a compound, or a salt thereof, of the formula (V):

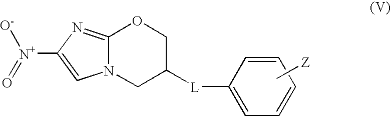

wherein Land Z are as defined above for the compound of formula (II) and wherein each ring can independently be further substituted by 1, 2, 3 or more substituents, e.g. selected from the group consisting of lower alkyl, halogen, hydroxy, amino or lower alkoxy. In a preferred embodiment, L is selected from the group consisting of —OCH₂C(O)—, —OCH₂C(O)NH—, —OCH₂C(O)NHCH₂— and —OCH₂CH₂— and Z is selected from the group consisting of —F, —CF₃, or —OCF₃. Preferably, Z is in the 3-position, more preferably in the 4-position.

In another aspect, the present invention provides a pharmaceutical composition comprising a compound of formula (I), (II) or (V), or a compound of any of formulae (Ia) to (Ii) as defined above, or a pharmaceutically acceptable salt, ester or prodrug thereof in combination with a pharmaceutically acceptable excipient, diluent or carrier.

In another aspect, the present invention provides a compound of formula (I), (II) or (V), or a compound of any of formulae (Ia) to (Ii) as defined above, or a pharmaceutically acceptable salt, ester or prodrug thereof for use as a medicine.

In another aspect, the present invention provides a method of treating and/or preventing a disease caused by a pathogenic microbe or a parasite such as Mycobacterium tuberculosis, Trypanosoma cruzi or Leishmania donovani comprising administering to a human or animal subject in need thereof a therapeutically effective amount of a compound of formula (I), (II) or (V), or a compound of any of formulae (Ia) to (Ii) as defined above, or a pharmaceutically acceptable salt, ester or prodrug thereof.

In yet another aspect, the present invention provides a pharmaceutical composition comprising a compound of formula (I), (II) or (V), or a compound of any of formulae (Ia) to (Ii) as defined above, or a pharmaceutically acceptable salt, ester or prodrug thereof, for the manufacture of a medicament for the treatment and/or prevention of a disease caused by a pathogenic microbe such as Mycobacterium tuberculosis.

In yet another aspect, the present invention provides the use of a compound of formula (I), (II) or (V), or a compound of any of formulae (Ia) to (Ii) as defined above, or a pharmaceutically acceptable salt, ester or prodrug thereof, for the manufacture of a medicament for the treatment and/or prevention of a disease caused by a pathogenic microbe such as Mycobacterium tuberculosis.

Preferably the disease is TB, more preferably multi-drug resistant TB.

In another aspect, the present invention provides a method of treating and/or preventing a disease or disorder caused by an infection by Trypanosoma cruzi or Leishmania donovani, comprising administering to a human or animal subject in need thereof an effective amount of a compound of formula (III), or a pharmaceutically acceptable salt, ester or prodrug thereof.

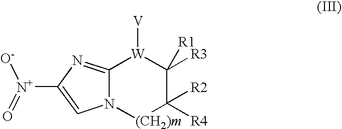

wherein:
(a) m is 0;
   W is O and V is absent;
   one of R1 and R3 is haloaryl or alkyl and the other is H, or R1 and R3 are both a lower alky group; and
   R2 and R4 are both H;
or:
(b) m is 1
   W is N and V is an alkylaryl group, optionally substituted with one or more alkoxy substituents;
   R1 and R3 are both H; and
   One of R2 and R4 is alkoxy and the other is H;
or:
(c) m is 1;
   W is O and V is absent;
   one of R1 and R3 is alkyl or aryl, and the other is H; and
   R2 and R4 are both H;
or:
(d) m is 1;
   W is O and V is absent;
   one of R2 and R4 is -L(B)ₙ-(Z)ₚ, -(L-B)_{q}-(Z)ₚ or —Y-(B)_{q}-Z, and the other is H; and
   R1 and R3 are both H;
   wherein L is an atom group having of the formula —O—R5 where R5 is a lower alkylene, —C(O)ₙ—, lower alkylene-C(O)—, —C(O)-lower alkylene, lower alkylene-C(O)—NH—, lower alkylene-NH—; B is a cycloalkyl, heterocyclic, aryl or heteroaryl ring which is optionally further substituted with one or more substituents; and Z is halogen, lower alkyl substituted with at least one halogen, lower alkoxy substituted with at least one halogen or lower thioalkyl substituted with at least one halogen;
   and Y is —NHC(O)—;
   n is 1 or 2; p is 0, 1 or 2; and q is 1 or 2.

In yet another aspect, the present invention provides a pharmaceutical composition comprising a compound of formula (I), (II), (II) or (V), or a compound of any of formulae (Ia) to (Ii) as defined above, or a pharmaceutically acceptable salt, ester or prodrug thereof, for the manufacture of a medicament for the treatment and/or prevention of a disease caused by a parasite such as Trypanosoma cruzi or Leishmania donovani.

In yet another aspect, the present invention provides the use of a compound of formula (I), (II), (III) or (V), or a compound of any of formulae (Ia) to (Ii) as defined above, or a pharmaceutically acceptable salt, ester or prodrug thereof, for the manufacture of a medicament for the treatment and/or prevention of a disease caused by a parasite such as Trypanosoma cruzi or Leishmania donovani.

Preferably the disease is Chagas disease or Leishmaniasis.

In another aspect, the present invention provides a compound of formula (I), (II), (III) or (V), or a compound of any of formulae (Ia) to (Ii) as defined above, or a pharmaceutically acceptable salt, ester or prodrug thereof for use in combination with a first line or a second line TB drug. In still another aspect, the present invention provides a combination comprising a) a compound of formula (I), (II), (III) or (V), or a compound of any of formulae (Ia) to (Ii) as defined above, or a pharmaceutically acceptable salt, ester or prodrug thereof and b) at least one TB drug selected from the group consisting of Isoniazid, Rifampicin, Pyrazinamide, Ethambutol, Streptomycin, Capreomycin, Kanamycin, Ethioamide, Para-aminosalicylic acid (PAS), Cycloserine, Ciprofloxacin, Ofloxacin, Amikacin, Clofazimine, Thiacetazone, Gatifloxacin, Moxifloxacin.

In still another aspect the invention provides a method for the preparation of a nitrogen heterocyclic compound, wherein the method comprises:

reacting a non-sterically hindered substituted epoxide with a haloimidazole compound, wherein the molar ratio of the non-sterically hindered substituted epoxide to the haloimidazole compound is less than or equal to 1:1, to form an adduct with an alcohol functional group;
protecting the alcohol functional group on the adduct to form an alcohol-protected adduct; and
treating the alcohol-protected adduct with a cyclizing agent to form the nitrogen heterocyclic compound.

Preferably the nitrogen heterocyclic compound, in free or salt form, is represented by a compound of formula (IV)

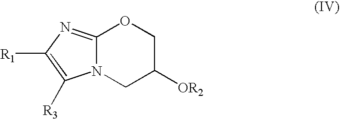

wherein R₁=nitro, acyl, formyl, sulfonyl, trifluoromethyl, cyano, halo or alkoxycarbonyl; R₂=2-tetrahydropyranyl, 2-ethoxyethyl, trityl, methyl, ethyl, allyl, trimethylsilyiethoxymethyl, 2,2,2-trichloroethyl, benzyl, trimethylsilyl, t-butyldimethylsilyl, phenyldimethylsilyl, triisopropylsilyl or thexyldimethylsilyl; R₃=H, acyl, formyl, sulfonyl, trifluoromethyl, cyano, halo or alkoxycarbonyl.

Preferably the non-sterically hindered substituted epoxide further comprises a masked alcohol moiety. It is further preferred that the non-sterically hindered substituted epoxide is represented by the compound of formula (Ij)

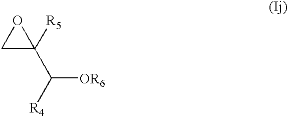

wherein R₄=H, alkyl, alkenyl, aryl, heteroalkyl, heteroalkenyl or heteroaryl; R₅=H, alkyl, alkenyl, aryl, heteroalkyl, heteroalkenyl or heteroaryl; R=trimethylsilyl, triethylsilyl, t-butyldimethylsilyl, dibutylmethylsilyl, diphenylmethylsilyl, phenyldimethylsilyl or diphenyl-t-butylsilyl.

It is further preferred that the haloimidazole compound is represented by a compound of formula (Ik)

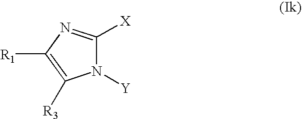

wherein X═Cl, Br or I; Y═H, Li, Na, K, CO₂H, CO₂—, t-butoxycarbonyl, N,N-dimethylaminosulfonyl, p-toluenesulfonyl, or triisopropylsilyl; R₁=nitro, acyl, formyl, sulfonyl, trifluoromethyl, cyano, halo or alkoxycarbonyl; R₃=H, acyl, formyl, sulfonyl, trifluoromethyl, cyano, halo or alkoxycarbonyl.

It is still further preferred that the alcohol-protected adduct is represented by a compound of formula (Im)

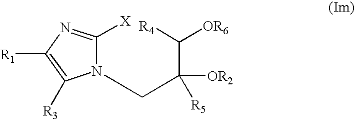

wherein R₁=nitro, acyl, formyl, sulfonyl, trifluoromethyl, cyano, halo or alkoxycarbonyl; R₂=2-tetrahydropyranyl, 2-ethoxyethyl, trityl, methyl, ethyl, allyl, trimethylsilylethoxymethyl, 2,2,2-trichloroethyl, benzyl, trimethylsilyl, t-butyldimethylsilyl, phenyldimethylsilyl, triisopropylsilyl or thexyldimethylsilyl; R₃=H, acyl, formyl, sulfonyl, trifluoromethyl, cyano, halo or alkoxycarbonyl; R₄=H, alkyl, alkenyl, aryl, heteroalkyl, heteroalkenyl or heteroaryl; R₅=H, alkyl, alkenyl, aryl, heteroalkyl, heteroalkenyl or heteroaryl; R₆=H, trimethylsilyl, triethylsilyl, t-butyldimethylsilyl, dibutylmethylsilyl, diphenylmethylsilyl, phenyldimethylsilyl or diphenyl-t-butylsilyl; X=Cl, Br or I.

Preferably the molar ratio of the non-sterically hindered substituted epoxide to the haloimidazole compound is in the range of 0.55 to 0.95:1, more preferably in the range of 0.6 to 0.9:1, still more preferably in the range of 0.65 to 0.85:1, still more preferably in the range of 0.65 to 0.8:1, still more preferably in the range of 0.7 to 0.85:1, still more preferably in the range of 0.7 to 0.8:1.

Preferably the non-sterically hindered substituted epoxide is reacted with a haloimidazole compound, to form the adduct with an alcohol functional group at a temperature range of 45-105° C., more preferably 55-95° C., still more preferably 65-85° C., still more preferably 60-80° C.

Preferably the haloimidazole compound contains a halogen substituent selected from the group consisting of chloro or bromo.

It is further preferred that the alcohol functional group on the adduct is treated to form the alcohol-protected adduct in the presence of a catalyst.

Preferably the catalyst is pyridinium-p-toluene sulfonate. It is also preferred that the cyclizing agent is selected from the group consisting of anhydrous TBAF, anhydrous TBABr or NaH.

Preferably the alcohol-protected adduct is treated with a cyclizing agent to form the nitrogen heterocyclic compound under microwave conditions.

Preferably the primary unprotected alcohol adduct is treated with a cyclizing agent to form the nitrogen heterocyclic compound under microwave conditions.

It is also preferred that the alcohol-protected adduct is treated with a cyclizing agent to form the nitrogen heterocyclic compound in vacuo.

Preferably R₂ is tetrahydropyranyl. Preferably R₃=H. Preferably R₄ and R₅ are independently selected from the group consisting of hydrogen and alkyl. Preferably R₆ is t-butyldimethylsilyl (TBDMS).

More preferably R₃, R₄, and R₅ are all H.

Preferably the nitrogen heterocyclic compound is a 3-alkyloxy-6-nitro-2H-3,4-dihydro-[2-1 b]imidazopyran or a 3-aryloxy-6-nitro-2H-3,4-dihydro-[2-1b]imidazopyran. More preferably the 3-alkyloxy-6-nitro-2H-3,4-dihydro-[2-1 b]imidazopyran or the 3-aryloxy-6-nitro-2H-3,4-dihydro-[2-1b]imidazopyran is an (S)- or an (R)-isomer.

In one preferred embodiment the nitrogen heterocyclic compound is 3(S)-tetrahydropyranyloxy-6-nitro-2H-3,4-dihydro-[2-1 b]imidazopyran.

In another preferred embodiment the nitrogen heterocyclic compound is 3(R)-tetrahydropyranyloxy-6-nitro-2H-3,4-dihydro-[2-1 b]imidazopyran.

The invention further provides a method for the preparation of a nitrogen heterocyclic compound, wherein an alcohol-protected adduct represented by the compound of formula (In)

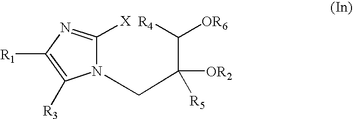

wherein R₁=nitro, acyl, formyl, sulfonyl, trifluoromethyl, cyano, halo or alkoxycarbonyl; R₂=2-tetrahydropyranyl, 2-ethoxyethyl, trityl, methyl, ethyl, allyl, trimethylsilylethoxymethyl, 2,2,2-trichloroethyl, benzyl, trimethylsilyl, t-butyldimethylsilyl, phenyldimethylsilyl, triisopropylsilyl or thexyldimethylsilyl; R₃=H, acyl, formyl, sulfonyl, trifluoromethyl, cyano, halo or alkoxycarbonyl; R₄=H, alkyl, alkenyl, aryl, heteroalkyl, heteroalkenyl or heteroaryl; R₅=H, alkyl, alkenyl, aryl, heteroalkyl, heteroalkenyl or heteroaryl; R₆=trimethylsilyl, triethylsilyl, t-butyldimethylsilyl, dibutylmethylsilyl, diphenylmethylsilyl, phenyldimethylsilyl or diphenyl-t-butylsilyl and R₆=H;
is treated with a cyclizing agent, under microwave conditions to form a nitrogen heterocyclic compound represented by the formula (IV).

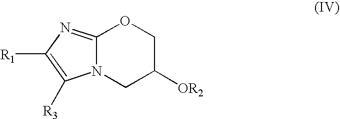

Preferably the cyclizing agent is selected from the group consisting of anhydrous TBAF, anhydrous TBABr or NaH. It is also preferred that the alcohol-protected adduct is treated under microwave conditions under elevated pressure.

It is further preferred that the primary unprotected alcohol adduct is treated under microwave conditions under elevated pressure.

Preferably the nitrogen heterocyclic compound is an (R)- or (S)-isomer.

The invention also provides a method as described above, further comprising reacting a compound represented by the formula (IV)

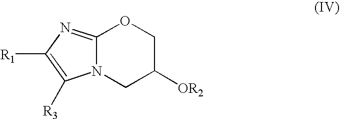

wherein R₁=nitro; R₂=2-tetrahydropyranyl, 2-ethoxyethyl, trityl, methyl, ethyl, allyl, trimethylsilylethoxymethyl, 2,2,2-trichloroethyl, benzyl, trimethylsilyl, t-butyldimethylsilyl, phenyldimethylsilyl, triisopropylsilyl or thexyldimethylsilyl; R₃=H; with 4-(trifluoromethoxy)benzyl halide
to form a nitrogen heterocyclic compound represented by the formula (Io)

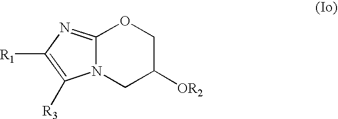

wherein R₁=nitro; R₂=trifluoromethoxybenzyl; R₃=H.

Preferably the 4-(trifluoromethoxy)benzyl halide is selected from the group of consisting of 4-(trifluoromethoxy)benzyl bromide, 4-(trifluoromethoxy)benzyl chloride and 4-(trifluoromethoxy)benzyl iodide.

Preferably the compound represented by the formula (IV)

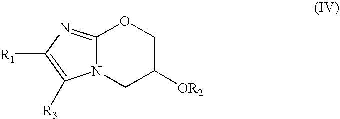

wherein R₁=nitro; R₂=2-tetrahydropyranyl, 2-ethoxyethyl, trityl, methyl, ethyl, allyl, trimethylsilylethoxymethyl, 2,2,2-trichloroethyl, benzyl, trimethylsilyl, t-butyldimethylsilyl, phenyldimethylsilyl, triisopropylsilyl or thexyldimethylsilyl; R₃=H;
is further treated with an alcohol-deprotecting agent before reacting with 4-(trifluoromethoxy)benzyl halide. Preferably the alcohol-deprotecting agent is selected from the group comprising of acetic acid, TBAF, TBABr.

It is further preferred that the nitrogen heterocyclic compound is an (R)- or (S)-isomer.

### DETAILED DESCRIPTION

### Definitions

The term “alkyl” as used herein includes both straight chain and branched alkyl groups. Alkyl comprises preferably 1 to 8 carbon atoms. Any alkyl, alkoxy, alkylene, cycloalkyl, heterocyclic residue, aryl or heteroaryl may be, unless otherwise stated, unsubstituted or substituted by one or more substituents selected from e.g. lower alkyl, halogen, hydroxy, amino. The term “alkylene” refers to a divalent radical derived from alkyl.

The term “lower alkyl” as used herein refers to branched or straight chain alkyl groups comprising 1 to 5 carbon atoms, preferably 1 to 3 carbon atoms such as e.g., methyl, ethyl, propyl, isopropyl, n-propyl, n-butyl, sec-butyl, t-butyl. The term “lower alkoxy” as used herein refers to —OR wherein R is lower alkyl as defined above. Examples of lower alkoxy groups include e.g. methoxy, ethoxy, t-butoxy.

The term “alkenyl” as used herein includes straight chain or branched alkenyl, which may be, for example, C₂-C₁₂ alkenyl in all its isomeric forms.

The term “alkoxycarbonyl” means a group RCO wherein R is an alkoxy group, for example, a C₁-C₁₂-alkoxy group, in all its isomeric forms.

“Halo” or “halogen” means F, Cl, Br or I, preferably F or Cl.

The term “alkyl halogen” or “haloalkyl” refers to an alkyl group as defined above to which at least one halogen as defined above is attached. Examples are e.g. fluoromethyl, difluoromethyl, trifluoromethyl, pentafluoroethyl. The term “lower alkyl halogen” or “lower haloalkyl” has a corresponding meaning to the term “(lower alkyl” as defined above.

The term “lower alkoxy halogen” or “lower haloalkoxy” refers to a lower alkoxy group as defined above to which at least one halogen as defined above is attached. Examples are e.g. fluoromethoxy, difluoromethoxy, trifluoromethoxy, pentafluoroethoxy.

The term “cycloalkyl” refers to a saturated or partially saturated (non-aromatic) cyclic ring which is optionally further substituted, e.g. with lower alkyl, halogen, hydroxy, amino. Examples include e.g. cyclopentyl, cyclohexyl, methylcyclohexyl. The cycloalkyl ring is preferably a 5 or 6 membered cyclic ring.

The term “aryl” refers to an aromatic monocyclic or fused bicyclic ring structure which can contain from 4 to 12 carbon atoms, preferably 5 or 6 carbon atoms for monocyclic rings and 8, 9 or 10 carbon atoms for fused bicyclic rings. The aryl group is optionally further substituted, e.g. with lower alkyl, halogen, hydroxy, amino. An aryl group may be e.g. phenyl or naphthyl, preferably phenyl. The term “haloaryl” means an aryl group substituted with one or more halogens as defined above, preferably one or more fluoro groups. The term “alkylaryl” means —R-aryl where R is an alkyl group as defined above and aryl is as defined above. An example is benzyl.

The term “heterocyclic” refers to a saturated or partially saturated (non-aromatic) ring which comprises additionally 1, 2 or 3 heteroatoms selected from the group consisting of N, O and S, and which is optionally condensed to 1 or 2 benzene rings and/or to a further heterocyclic ring and optionally further substituted on a ring C or ring heteroatom with e.g. a lower alkyl, halogen, hydroxy, amino group.

The term “heteroaryl” refers to an aromatic heterocyclic ring, e.g. a 5 or 6 membered aromatic heterocyclic ring, optionally condensed to 1 or 2 benzene rings and/or to a further heterocyclic ring and optionally further substituted on a ring C or ring heteroatom with e.g. a lower alkyl, halogen, hydroxy, amino group. Examples of heterocyclic and heteroaryl groups include e.g. morpholinyl, piperazinyl, piperidinyl, pyridyl, pyrrolidinyl, pyrazinyl, pyrimidinyl, purinyl, pyranyl, benzimidazolyl, benzoxazolyl, benzthiazolyl, indolyl, indolinyl, quinolinyl, isoquinolinyl, 1,2,3,4-tetrahydroquinolinyl, thiazolyl, imidazolyl, benzotriazolyl, indanyl, oxadiazolyl, pyrazolyl, triazolyl, or tetrazolyl.

The term “nitrogen heterocyclic compound” as used herein refers to a cyclic structure containing an sp²-hybridized nitrogen in a ring of the structure.

As used herein, the term “substituted” is contemplated to include all permissible substituents of organic compounds, including cationic or anionic salt formation. In a broad aspect, the permissible substituents include acyclic and cyclic, branched and unbranched, carbocyclic and heterocyclic, aromatic and nonaromatic substituents of organic compounds. This invention is not intended to be limited in any manner by the permissible substituents of organic compounds.

A “non-sterically hindered substituted epoxide” refers to substituted epoxides where the substituents do not sterically hinder the underlying reaction and includes the implicit proviso that the substitution is in accordance with permitted valence of the substituted atom.

The term “substituted nitroimidazole” as used herein refers to an imidazole nucleus which carries both a nitro substituent as well as another substituent on the imidazole nucleus, commonly at an imidazole ring carbon position or at an imidazole ring nitrogen position.

The term “protecting group” as used herein refers to temporary or permanent chemical moieties which protect a potentially reactive functional group from unintended chemical transformations. Examples of such protecting groups include esters of carboxylic acids, silyl ethers of alcohols, and acetals and ketals of aldehydes and ketones, respectively. The field of protecting group chemistry is known in the art; examples of protecting groups include conventionally used protective groups which can be found, for example, in “Protective Groups in Organic Synthesis,” T. W. Greene, P. M. Wuts, John Wiley and sons 1991, pp. 10-142).

The term “masked alcohol moiety” as used herein refers to any group commonly used for the temporary protection of the hydroxyl functional group, resulting in masking the reactivity of the free alcohol group. Examples of suitable protecting groups for hydroxyl functional groups include but are not limited to the following examples: alkoxycarbonyl, acyl, alkylsilyl or alkylarylsilyl groups, and alkoxyalkyl groups.

The term “deprotecting” or “deprotection” as used herein is known in the art of organic synthesis and refers to conditions for the removal of protecting groups or chemical moieties that mask the underlying reactivity of the functional group, leaving the unprotected functional group. Examples of suitable deprotecting agents or conditions for various functional groups can be found, for example in “Protective groups in Organic Synthesis,” T. W. Greene, P. M. Wuts, John Wiley and sons 1991).

The term “free form” as used herein refers to non-salt forms of the compounds. Additionally, the compounds of the present invention can exist in unsolvated forms as well as solvated forms, including hydrated forms, crystalline forms and polymorphic forms.

The terms “salts” or “salt form” as used herein refers to base addition salts that are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, chbline, diethanolamine, dicyclohexylamine, ethylenediamine, N-methylglucamine, and procaine (see, for example, Berge S. M. et al., “Pharmaceutical Salts”, J. of Pharma. Sci., 66:1 (1977).

The term “electron-withdrawing group” is recognized in the art, referring to the tendency of a substituent to attract valence electrons from neighboring atoms, i.e. the substituent is electronegative with respect to neighboring atoms. Exemplary electron-withdrawing groups include nitro, acyl, formyl, sulfonyl, trifluoromethyl, cyano, halo, and the like.

The term “microwave conditions” as used herein refers to the use of technology used to produce or simulate microwave irradiation. Examples for the use of microwave irradiation in organic synthesis can be found, for example in Tetrahedron, 57:9225-9283 (2001) and Acc. Chem. Soc., 82:14-19 (2004).

The present invention also encompasses enantiomers, racemates, diastereoisomers and mixtures of the compounds of the invention. As is evident to those skilled in the art, the compounds of the invention contain asymmetric carbon atoms. It should be understood, therefore, that the individual stereoisomers are contemplated as being included within the scope of the invention. The terms “R” and “S” for chemical configuration as used herein, are as defined by IUPAC in “Recommendations for Section E, Fundamental Stereochemistry”, Pure Appl. Chem., 45:13-30 (1976).

The structures of the reagents and compounds identified herein by generic or tradenames may be taken, for example, from “The Merck Index” or from databases, e.g. Patents International (e.g. IMS World Publications) and accordingly, is enabling to any person skilled in the art.

### Compounds of the Invention

The compounds of the present invention are useful in the treatment and/or prevention of infections by a pathogen. The pathogen is preferably a bacterium or a protozoan, in particular a Mycobacterium, Clostriduim, Cryptosporidium, Helicobacter, Trypanosoma, Leishmania or Plasmodium. More specifically the bacterium or protozoan can be Mycobacterium tuberculosis (in particular multi-drug resistant Mycobacterium tuberculosis), Clostridium difficile, Cryptosporidium parvum, Helicobacter pylori, T. brucei rhodesiense, T. brucei gambience, Trypanosoma cruzi, Leishmania donovani, L. major, Plasmodium falciparum, Mycobacterium avium, Mycobacterium ulcerans. In particular, the pathogen is Mycobacterium tuberculosis, Trypanosoma cruzi or Leishmania donovani.

The compounds of the present invention, in free form or in pharmaceutically acceptable salt form, exhibit valuable pharmacological properties, e.g. as antimicrobial agents, for example, as indicated by the tests of the Examples and are therefore indicated for therapy.

The compounds of the present invention exhibit IC₅₀ against Leishmania donovani that are below 5 μM, preferably below 4 μM, more preferably below 3 μM, even more preferably below 2 μM, even more preferably below 1 μM, even more preferably below 0.5 μM and still more preferably below 0.1 μM.

The compounds of the present invention exhibit IC₅₀ against Trypanosoma cruzi that are below 5 μM, preferably below 4 μM, more preferably below 3 μM, even more preferably below 2 μM, even more preferably below 1 μM and still more preferably below 0.5 μM.

The compounds show a MIC against Mycobacterium tuberculosis that is preferably lower than 0.8 mM, more preferably lower than 0.5 μM, more preferably lower than 0.1 μM, more preferably lower than 0.05 μM, more preferably lower than 0.01 μM, more preferably lower than 0.005 μM, more preferably lower than 0.001 μM, more preferably lower than 0.0005

The compounds of the present invention may exist in free form or in salt form, e.g. addition salts with e.g. organic or inorganic acids, for example trifluoroacetic acid or hydrochloride acid, or salts obtainable when they comprise a carboxy group, e.g. with a base, for example alkali salts such as sodium, potassium, or substituted or unsubstituted ammonium-salts.

The compounds of the present invention may be administered as the sole active ingredient or as one active component in a combination tablet containing several active components, e.g. antibiotics.

The required dosage for pharmaceutical use will of course vary depending on the mode of administration, the particular condition to be treated and the effect desired. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, for example, in divided doses up to four times a day or in retard form. Suitable unit dosage forms for oral administration comprise from ca. 1 to 100 mg active ingredient.

The compounds of the present invention can be administered by any conventional route, in particular enterally, for example, orally, e.g. in the form of tablets or capsules, or parenterally, for example, in the form of injectable solutions or suspensions, topically, e.g. in the form of lotions, gels, ointments or creams, or in a nasal or a suppository form. Pharmaceutical compositions comprising a compound of formula I to 8 in free form or in pharmaceutically acceptable salt form in association with at least one pharmaceutical acceptable carrier or diluent can be manufactured in conventional manner by mixing with a pharmaceutically acceptable carrier or diluent.

The compounds of the present invention can be administered in free form or in pharmaceutically acceptable salt form, for example, as indicated above. Such salts can be prepared in a conventional manner and exhibit the same order of activity as the free compounds.

Compounds of formula (I) and (ii) can be prepared according to the following reaction scheme

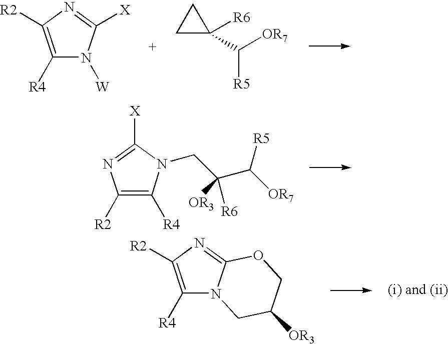

wherein R₂ is nitro, acyl, formyl, sulfonyl, trifluoromethyl, cyano, halo or alkoxycarbonyl; R₃ is a protection group, e.g. 2-tetrahydropyranyl, 2-ethoxyethyl, trityl, methyl, ethyl, allyl, trimethylsilylethoxy methyl, 2,2,2-trichloroethyl, benzyl, trimethylsilyl, t-butyldimethylsilyl, phenyldimethylsilyl, triisopropylsilyl or thexyldimethylsilyl, triethylsilyl; R₄ is H, acyl, formyl, sulfonyl, trifluoromethyl, cyano, halo or alkoxycarbonyl; R₅ is H, alkyl, alkenyl, aryl, heteroalkyl, heteroalkenyl or heteroaryl; R₆ is H, alkyl, alkenyl, aryl, heteroalkyl, heteroalkenyl or heteroaryl; R₇ is a protection group, e.g. trimethylsilyl, triethylsilyl, t-butyldimethylsilyl, dibutylmethylsilyl, diphenylmethylsilyl, phenyldimethylsilyl or diphenyl-t-butylsilyl; X is Cl, Br or I; W is H, Li, Na, K, CO₂H, CO₂⁻, t-butoxycarbonyl, N,N-dimethylaminosulfonyl, p-toluenesulfonyl, or triisopropylsilyl.

The present invention provides a method for the preparation of a nitrogen heterocyclic compound. In one embodiment the method comprises reacting a non-sterically hindered substituted epoxide with a haloimidazole compound, wherein the molar ratio of the non-sterically hindered substituted epoxide to the haloimidazole compound is less than or equal to 1:1, to form an adduct with an alcohol functional group; protecting the alcohol functional group on the adduct to form an alcohol-protected adduct; and treating the alcohol-protected adduct with a cyclizing agent to form the nitrogen heterocyclic compound. The present invention provides, furthermore a method to convert the bisprotected alcohol adduct to the mono deprotected primary alcohol adduct which is reacted with a cyclizing agent to form the nitrogen heterocyclic compound.

According to the method provided in the present invention, the use of explosive dinitroimidazoles are circumvented altogether by the use of haloimidazoles containing an electron-withdrawing substituent. Advantageously also, the method of the present invention provides an efficient method to the preparation of imidazopyrans while requiring fewer labour-intensive purification steps. The overall process is more efficacious and more amenable to large-scale synthesis than, for example, the method disclosed in U.S. Pat. No. 6,087,358.

By using an appropriate molar ratio of the starting materials in one of the steps of the synthesis, the step-wise yield as well as the overall yield of the synthesis may be increased. Additionally, with the use of an appropriate molar ratio of the starting materials, the laborious need to purify the product formed is obviated. The method of this invention may further expedite the efficiency of synthesis by incorporating a cyclization step, to form the nitroimidazopyran product in high yield. Cyclization may be effected in preferred embodiments under microwave conditions, under elevated pressure or a combination of both microwave conditions and elevated pressure.

Cyclization may be effected in preferred embodiments under microwave conditions, under elevated pressure or a combination of both microwave conditions and elevated pressure. The term “microwave; conditions” as used herein refers to the use of technology used to produce or simulate microwave irradiation. Examples for the use of microwave irradiation in organic synthesis can be found, for example in Tetrahedron, 57:9225-9283 (2001) and Acc. Chem. Soc., 82:14-19 (2004).

Thus in a preferred embodiment, the present invention provides a process for the conversion of compound (Ir) to compound (IV) using microwave irradiation.

In a preferred embodiment, the haloimidazole compound is represented by a compound of formula (Ip)

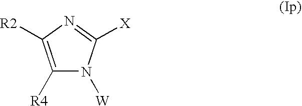

wherein X is Cl, Br or I; W is H, Li, Na, K, CO₂H, CO₂—, a protection group, e.g. t-butoxycarbonyl, N,N-dimethylaminosulfonyl, p-toluenesulfonyl, or triisopropylsilyl; R₂ is nitro, acyl, formyl, sulfonyl, trifluoromethyl, cyano, halo or alkoxycarbonyl; R₄ is H, acyl, formyl, sulfonyl, trifluoromethyl, cyano, halo or alkoxycarbonyl.

The haloimidazole compound may be activated for reaction by pre-treatment with an aprotic weak base such as anhydrous potassium carbonate, proton sponge, DBU and the like. The haloimidazole compound is reacted with a non-sterically hindered substituted epoxide in the presence of an anhydrous solvent such as anhydrous ethanol, anhydrous methanol, anhydrous THF, anhydrous N,N-dimethylformamide, anhydrous dichloromethane and the like. Nucleophilic addition of the haloimidazole compound, in free or salt form, to the non-sterically hindered substituted epoxide, using appropriate molar ratios, yields an adduct with an alcohol functional group.

The haloimidazole compound is a nitrogen heterocyclic compound containing the imidazole nucleus which carries a halogen substituent such as chloro, bromo, iodo and the like. The imidazole nucleus may be further substituted, commonly at the carbon or nitrogen ring atoms. Substituents in the representative classes such as acyl, formyl, sulfonyl, silyl, trifluoromethyl, cyano, halo, nitro or alkoxycarbonyl may be used.

In one aspect of the invention, the substituent at a carbon ring atom includes an electron-withdrawing group such as acyl, formyl, sulfonyl, trifluoromethyl, cyano, halo, nitro or alkoxycarbonyl. In a preferred embodiment, the substituent at an imidazole ring carbon atom is a nitro group. In a more preferred embodiment, the substituent is a nitro group in the 4-position on the imidazole nucleus.

Another aspect of the invention is the substitution at a ring nitrogen atom on the imidazole nucleus. The imidazole nitrogen atom may be unsubstituted, i.e. protonated, or derivatised such that the nitrogen atom is deprotonated or transiently protected so as to permit nucleophilic reaction with the epoxide compound. Examples of substituents at the nitrogen atom on the imidazole nucleus include, but are not limited to Li, Na, K, CO₂H, C₂⁻, t-butoxycarbonyl, N,N-dimethylaminosulfonyl, p-toluenesulfonyl, and triisopropylsilyl. The non-sterically hindered substituted epoxide is an epoxide containing substituents that do not sterically hinder the reaction of the epoxide with the haloimidazole. Examples of substituents on the epoxide include but are not limited to alkyl, alkenyl, aryl, heteroalkyl, heteroalkenyl, heteroaryl, including isomeric and stereoisomeric substituents. Substituents on the epoxide may be in any configuration and may give rise to symmetrically- or asymmetrically-substituted epoxides.

One aspect of the invention provides for substituents with functional groups such as hydroxyl, silyl, alkbxy, alkenyl, aryl, aryloxy, amino, cyano, acyl and groups of the like which do not pose a steric hindrance to the reaction with the haloimidazole. Reactive functional groups on the substituents may be protected such that the reactivity is masked. In one embodiment, the non-sterically hindered substituted epoxide further comprises a masked alcohol moiety. Representative hydroxyl protecting groups include acyl groups, benzyl and trityl ethers, tetrahydropyranyl ethers, trialkylsilyl ethers and allyl ethers.

In one embodiment of the invention, substituents on the epoxide may include silyl-protecting groups such as trimethylsilyl, triethylsilyl, t-butyldimethylsilyl, dibutylmethylsilyl, diphenylmethylsilyl, phenyldimethylsilyl, diphenyl-t-butylsilyl and analogous alkylated silyl radicals. In another embodiment, the non-sterically hindered substituted epoxide further comprises a masked amine moiety. Representative amino protecting groups include, formyl, acetyl, trifluoroacetyl, benzyl, benzyloxycarbonyl, t-butoxycarbonyl, trimethyl silyl, 2-trimethylsilyl-ethanesulfonyl, trityl and substituted trityl groups, allyloxycarbonyl, 9-fluorenylmethyloxycarbonyl, nitro-veratryloxycarbonyl, and the like.

In a preferred embodiment, the non-sterically hindered substituted epoxide is represented by the compound of formula (Iq)

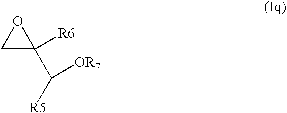

wherein R₅ is H, alkyl, alkenyl, aryl, heteroalkyl, heteroalkenyl or heteroaryl; R₆ is H, alkyl, alkenyl, aryl, heteroalkyl, heteroalkenyi or heteroaryl; R₇ is trimethylsilyl, triethylsilyl, t-butyldimethylsilyl, dibutylmethylsilyl, diphenylmethylsilyl, phenyldimethylsilyl or diphenyl-t-butylsilyl.

One aspect of the invention relates to the use of a molar ratio of an amount of the non-sterically hindered substituted epoxide to the haloimidazole compound. In one embodiment, the molar ratio of the non-sterically hindered substituted epoxide is less than or equal to 1:1. In another embodiment, the molar ratio of the non-sterically hindered substituted epoxide to the haloimidazole compound is in the range of 0.55 to 0.95:1. In another embodiment, the molar ratio of a non-sterically hindered substituted epoxide to haloimidazole compound is in the range of 0.6 to 0.9:1. In another embodiment, the molar ratio of a non-sterically hindered substituted epoxide to the haloimidazole compound is in the range of 0.65 to 0.85:1. In another embodiment, the molar ratio of a non-sterically hindered substituted epoxide to the haloimidazole compound is in the range of 0.65 to 0.8:1. In another embodiment, the molar ratio of a non-sterically hindered substituted epoxide to the haloimidazole compound is in the range of 0.7 to 0.85:1. In yet another embodiment, the molar ratio of a non-sterically hindered substituted epoxide to the haloimidazole compound is in the range of 0.7 to 0.8:1.

Another aspect of the invention relates to the reaction carried out at temperatures in the range of about 45-105° C. In a preferred embodiment, the non-sterically hindered substituted epoxide is reacted with a haloimidazole compound, to form the adduct with an alcohol functional group at a temperature range of about 55-95° C. In another preferred embodiment, the non-sterically hindered substituted epoxide is reacted with a haloimidazole compound, to form the adduct with an alcohol functional group at a temperature range of about 65-85° C. In yet another preferred embodiment, the non-sterically hindered substituted epoxide is reacted with a haloimidazole compound, to form the adduct with an alcohol functional group at a temperature range of about 60-80° C.

The haloimidazole compound reacts with a non-sterically hindered substituted epoxide to give a reaction product which, upon isolation in ways known to one skilled in the art, yields the adduct with the alcohol functional group as the end product. An example of the workup to form the end product includes filtration, removal of solvents, extraction between aqueous and organic phases using conventional organic solvents such as ethyl acetate, diethyl ether, chloroform, methylene chloride and the like, followed by drying the organic phase over conventional drying agents to give, upon removal of solvents, the adduct with an alcohol functional group.

The adduct formed by reaction of the non-sterically hindered epoxide with the haloimidazole contains an alcohol functional group derived by nucleophilic ring opening of the epoxide. The resulting adduct containing the alcohol functional group is isolated in its protonated form and is obtained with a purity greater than 90% and in yields equal to or greater than 90% based on the epoxide. Advantageously, the adduct with an alcohol functional group can be prepared on a large scale and is used in the subsequent step of the method without the need for further purification. The unreacted haloimidazole compound used as starting material in the reaction can be recovered from the aqueous layer and recycled for reaction, making the overall process even more cost-effective, efficient and amenable to large scale synthesis.

In one embodiment, the alcohol-protected adduct is represented by a compound of formula (Ir)

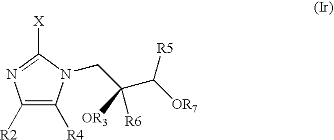

wherein R₂ is nitro, acyl, formyl, sulfonyl, trifluoromethyl, cyano, halo or alkoxycarbonyl; R₃ is a protection group, e.g. 2-tetrahydropyranyl, 2-ethoxyethyl, trityl, methyl, ethyl, allyl, trimethylsilylethoxymethyl, 2,2,2-trichloroethyl, benzyl, trimethylsilyl, t-butyldimethylsilyl, phenyldimethylsilyl, triisopropylsilyl or thexyldimethylsilyl, triethylsilyl, benzyloxycarcbonyl, allyloxycarbonyl; R₄ is H, acyl, formyl, sulfonyl; trifluoromethyl, cyano, halo or alkoxycarbonyl; R₅ is H, alkyl, alkenyl, aryl, heteroalkyl, heteroalkenyl or heteroaryl; R₆ is H, alkyl, alkenyl, aryl, heteroalkyl, heteroalkenyl or heteroaryl; R₇H, is a protection group, e.g. trimethylsilyl, triethylsilyl, t-butyldimethylsilyl, dibutylmethylsilyl, diphenylmethylsilyl, phenyldimethylsilyl, or diphenyl-t-butylsilyl; X is Cl, Br or I.

The stereochemistry of the method of the present invention is determined by the enantiomer selected for use for the non-sterically hindered substituted epoxide. Accordingly, the enantiomer afforded by the method of the present invention can be either the (S)- or the (R)-enantiomer, depending on the choice of the enantiomer used in the epoxide starting material.

The selective formation of regioisomers is another aspect of this invention. Depending on the direction of nucleophilic addition, one or the other regioisomer is isolated. According to the method of the present invention, only one regioisomer is favoured and is isolated from the reaction. The other regioisomer has been undetectable by the methods of detection using LC-MS and LC-UV spectroscopy. Another step of the method of the present invention comprises protecting the alcohol adduct, preferably in the presence of a catalyst, to form an alcohol-protected adduct. Methods for converting an alcohol to an alcohol-protected adduct are known in the art, particularly in the art of protecting groups, for example, in Chapter Two of “Protective Groups in Organic Synthesis”, T. W. Greene and P. G. M. Wuts, 3^{rd} ed. 1999. Accordingly, the method of this invention comprises the step of treating the alcohol adduct with a protecting group to convert the alcohol adduct to the corresponding alcohol-protected adduct. Examples of such alcohol protecting groups include but are not limited to dihydropyranyl-, 2-tetrahydropyranyl, 2-ethoxyethyl, trityl, methyl, ethyl, allyl, trimethylsilylethoxymethyl, 2,2,2-trichloroethyl, benzyl, trimethylsilyl, t-butyldimethylsilyl, phenyldimethylsilyl, triisopropylsilyl and thexyldimethylsilyl. In one embodiment, the 3,4-dihydro-2H-pyran is used to convert the alcohol adduct to the corresponding dihydropyranyl-protected alcohol adduct. Freshly-distilled 3,4-dihydro-2H-pyran obtained for example from a Kugelrohr distillation apparatus is preferred.

In transforming the adduct with an alcohol functional group to the corresponding alcohol-protected adduct, mild reaction conditions are employed to avoid cleavage of any reactive groups or transiently masked reactive groups derived from the non-sterically hindered epoxide. The mild conditions include stirring the reaction at room temperatures ranging from 15° C. to 35° C., for a time period of approximately 20-30 hours. To facilitate the reaction, particularly at lower temperatures, a catalyst may be added. Such catalysts are known in the art and include acyl groups, benzyl and trityl ethers, tetrahydropyranyl ethers, trialkylsilyl ethers and allyl ethers. However, the use of catalysts such as para-toluenesulfonic acid has been found to effect cleavage of the t-butylidimethylsilyl (TBDMS) protecting group. In a preferred embodiment, the catalyst is pyridinium-p-toluene sulfonate. At the end of the time period for the reaction between the alcohol adduct and the protecting group, the reaction may be stopped, for example, by quenching the reaction, with saturated aqueous sodium bicarbonate solution and the like. Upon removal of the organic layer, the aqueous layer is extracted several times with a volatile organic solvent such as dichloromethane, diethylether and ethyl acetate. The organic layers obtained from extraction are combined, washed with water, brine and dried over conventional drying agents such as magnesium sulfate. Removal of the solvents in vacuo gives a residue which is filtered over silica to remove any residual catalyst which adheres to the column. Elution with 50% EtOAc in hexanes and removal of the solvents in vacuo yields the alcohol-protected adduct which can be subsequently used without the need for further purification. Another aspect of the invention relates to the cyclization of the alcohol-protected adduct to form the nitrogen heterocyclic compound. In one embodiment, the alcohol-protected adduct is cyclized in the presence of a cyclizing agent, wherein the cyclizing agent is selected from the group consisting of anhydrous TBAF and anhydrous TBABr.

In another embodiment, the alcohol-protected adduct is treated with a cyclizing agent to form the nitrogen heterocyclic compound under microwave conditions. In another embodiment, the alcohol-protected adduct is treated with a cyclizing agent under elevated pressure to form the nitrogen heterocyclic compound. In a preferred embodiment of the invention, the alcohol-protected adduct is treated with a cyclizing agent to form the nitrogen heterocyclic compound under microwave conditions under elevated pressure. The group of cyclizing agents can further be extended for this reaction, including bases like Huenig's base, triethylamine, and the like and NaH.

In another embodiment, the primary unprotected alcohol adduct is treated with a cyclizing agent to form the nitrogen heterocyclic compound under microwave conditions. In another embodiment, the primary unprotected alcohol adduct is treated with a cyclizing agent under elevated pressure to form the nitrogen heterocyclic compound. In a preferred embodiment of the invention, the primary unprotected alcohol-protected adduct is treated with a cyclizing agent to form the nitrogen heterocyclic compound under microwave conditions under elevated pressure.

In the cyclization reaction, the alcohol-protected adduct is dissolved in an anhydrous aprotic solvent such as THF before addition of a cyclizing agent. Use of an autosampler to prepare several 20 to 30 mL reaction vessels containing anhydrous THF expedites as well as scales up the process of preparing large quantities of anhydrous THF for use. In a preferred embodiment, the cyclizing agent is anhydrous TBAF. Commercially available TBAF (Aldrich, 1 M, THF solution) is rendered anhydrous. In a further embodiment, anhydrous TBAF is degassed, preferably with nitrogen or argon, prior to use.

The reaction vessels containing the ether in anhydrous solvent as well as the cyclizing agent are sealed before exposure to microwave conditions under elevated pressure at a temperature range from 100° C. to 160° C., for a time period of 10-30 minutes. In one embodiment, microwave conditions can be generated using the Biotage system. (http://www.biotagedcg.com/) At the end of the time period, removal of the solvent from the reaction vessel yields a residue which upon purification on silica gel column chromatography, gives the desired nitrogen heterocyclic compound in yields equal or greater than 70%. By comparison, alternative synthetic routes for the synthesis of N-substituted-4-nitro-haloimidazoles and nitroimidazopyrans as disclosed in WO 2004/035547 report yields of about 50%.

The nitrogen heterocyclic compound formed is an imidazopyran carrying a alcohol functional group at the 3-position on the pyran ring. In one embodiment, the nitrogen heterocyclic compound, in free or salt form, is represented by a compound of formula (Ii)

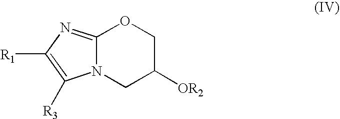

wherein R₁=nitro, acyl, formyl, sulfonyl, trifluoromethyl, cyano, halo or alkoxycarbonyl; R₂=2-tetrahydropyranyl, 2-ethoxyethyl, trityl, methyl, ethyl, allyl, trimethylsilylethoxymethyl, 2,2,2-trichloroethyl, benzyl, trimethylsilyl, t-butyldimethylsilyl, phenyldimethylsilyl, triisopropylsilyl or thexyldimethylsilyl; R₃=H, acyl, formyl, sulfonyl, trifluoromethyl, cyano, halo or alkoxycarbonyl.

In one embodiment, the nitrogen heterocyclic compound contains a nitro substituent at the 6-position. In another embodiment, the nitrogen heterocyclic compound formed is an (S)-isomer. In a preferred embodiment, the nitrogen heterocyclic compound formed is an (S)-isomer, with a nitro substituent at the 6-position.

Another aspect of the invention provides a method for the preparation of a nitrogen heterocyclic compound, wherein the nitrogen heterocyclic compound is an alkyloxy-6-nitro-2H-3,4-dihydro-[2-1b]imidazopyran or aryloxy-6-nitro-2H-3,4-dihydro-[2-1 b]imidazopyran. In one embodiment of the invention, a method is provided for the preparation of a nitrogen heterocyclic compound of formula (I), wherein the nitrogen heterocyclic compound is 3-alkyloxy-6-nitro-2H-3,4-dihydro-[2-1b]imidazopyran or the 3-aryloxy-6-nitro-2H-3,4-dihydro-[2-1]imidazopyran. In another embodiment, the nitrogen heterocyclic compound may be an (S)- or (R)-isomer. In one embodiment of the invention, a method is provided for the preparation of a nitrogen heterocyclic compound of formula (I), wherein the nitrogen heterocyclic compound is 3(R)-tetrahydropyranyloxy-6-nitro-2H-3,4-dihydro-[2-1b]imidazopyran. In a preferred embodiment of the invention, a method is provided for the preparation of a nitrogen heterocyclic compound of formula (I), wherein the nitrogen heterocyclic compound is 3(S)-tetrahydropyranyloxy-6-nitro-2H-3,4-dihydro-[2-1b]imidazopyran.

Another aspect of the invention further comprises converting a compound as represented by the formula (IV)

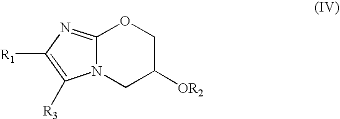

wherein R₁=nitro; R₂=2-tetrahydropyranyl, 2-ethoxyethyl, trityl, methyl, ethyl, allyl, trimethylsilylethoxymethyl, 2,2,2-trichloroethyl, benzyl, trimethylsilyl, t-butyldimethylsilyl, phenyldimethylsilyl, triisopropylsilyl or thexyldimethylsilyl; R₃=H, acyl, formyl, sulfonyl, trifluoromethyl, cyano, halo or alkoxycarbonyl;
to a compound as represented by the formula:

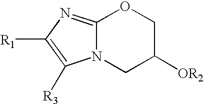

wherein R₁=nitro; R₂=trifluoromethoxybenzyl; R₃=H.

In another aspect the present invention provides a process for preparing the compounds of the present invention, comprising:

a) reacting a compound of formula (Is)

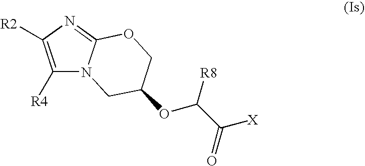

wherein R₂ is defined as above for compounds (Ip)-(Ir); R₄ is defined as above is defined as above for compounds (Ip)-(Ir); R₈ is H, alkyl, alkenyl, aryl, heteroalkyl, heteroalkenyl or heteroaryl and wherein X is Cl, Br, I, —OCOO-isobutenyl, lower alkyl, phenyl; with a compound of formula (It)

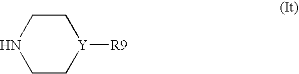

wherein Y is N or CH; R₉ is H, alkyl, alkenyl, aryl, heteroalkyl, heteroalkenyl, heteroaryl ortho-, meta- or para substituted trifluoro-, trifluoromethoxy-, fluororo-phenyl, biphenyl, heteroaryl, benzyl.
or with a compound of formula (Iw)

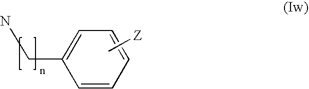

wherein Z is defined as above and n=0, 1, 2 or 3;
or with a compound of formula (Ix)

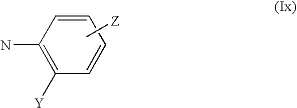

wherein Y and Z are defined as above;
or with a compound of formula (Iy)

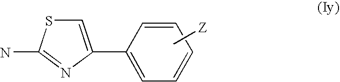

wherein Z is defined as above;
b) reacting compound of formula (Iz)

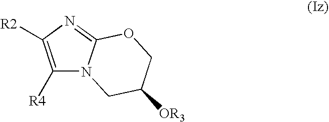

wherein R₂ is defined as above for compounds (Ip)-(Ir); R₄ is defined as above for compounds (Ip)-(Ir); and R₃ is H or a counterion as Li, Na, K, Mg, Zn, Ca;
with formula (Iaa)

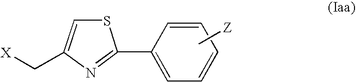

and wherein X is a halogen, Cl, Br, I;
or with formula (Iab)

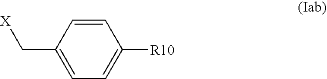

wherein R₁₀ is H, alkyl, alkenyl, aryl, heteroalkyl, fluoroalkyl, fluoroalkenyl, difluoroalky, trifluoroalkyl, pentafluoroethyl, hepafluoropropyl, nonafluorobutyl, heteroalkenyl, heteroaryl, ortho-meta- or para substituted trifluoro-, trifluoromethoxy-, fluororo-phenyl, biphenyl, heteroaryl, benzimidazolyl, benzothiazolyl, benzoxazolyl, benzyl and wherein X is a halogen, Cl, Br, I.

General Methods for Preparing Compounds of the Invention

Scheme 1 illustrates two important intermediates 6 and 7. Scheme 2 illustrates the preparation of a compound of formula (If), scheme 3 for a compound of formula (Ig), scheme 4 for a compound of formula (I), (ii), (Ia), (Ic), (Ie), scheme 5 and scheme 6 for a compound of formula (Id).

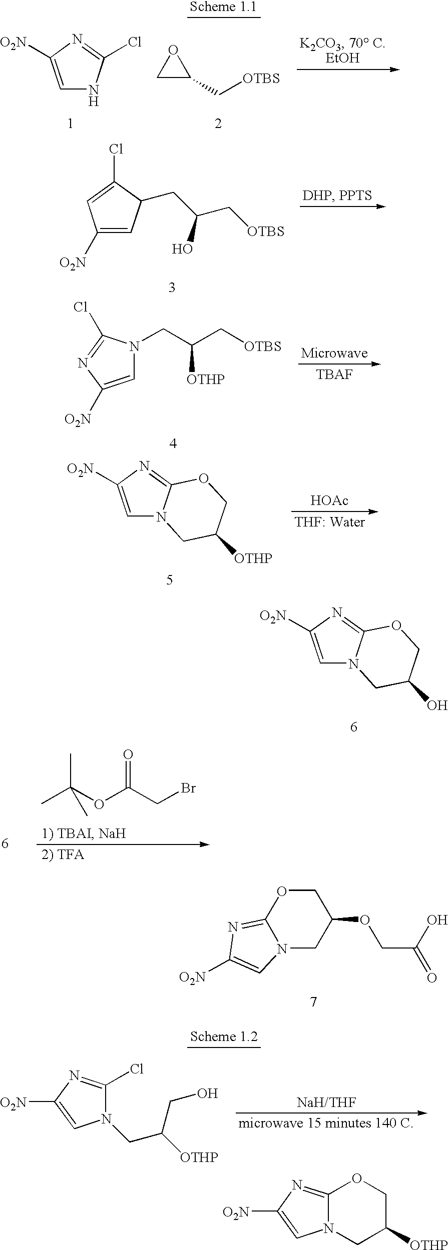

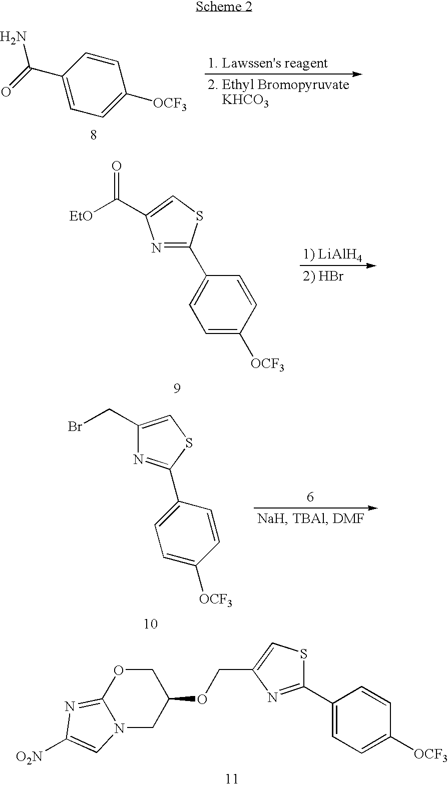

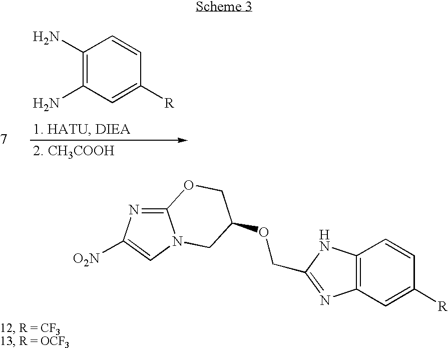

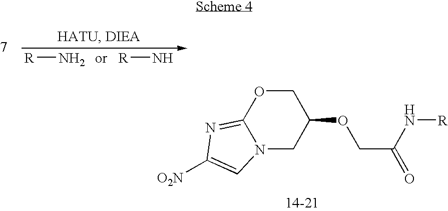

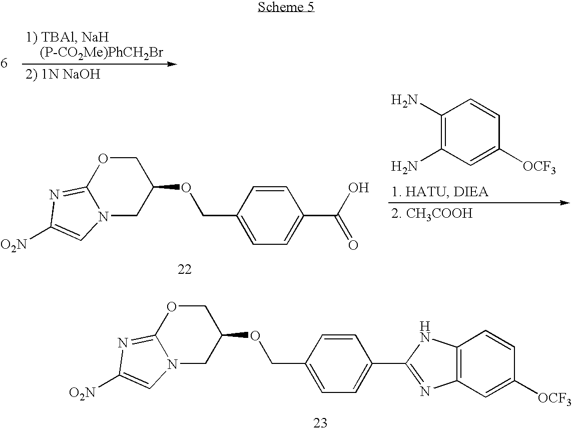

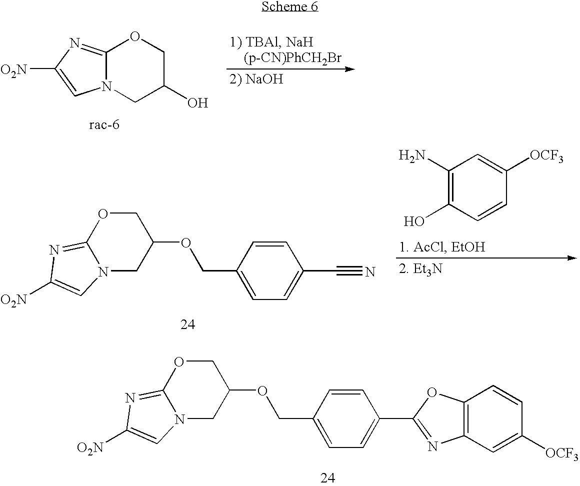

### EXAMPLES

The invention is described with reference to the following examples. It is to be appreciated that the invention is not limited to the examples.

### Example 1

### (S)-1-(tert-Butyl-dimethyl-silanyloxy)-3-(2-chloro-4-nitro-imidazol-1-yl)-propan-2-ol (3)

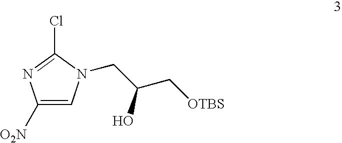

A mixture 2-chloro-4-nitroimidazole (20.0 g, 0.14 mol, 100 mol %) is dissolved in anhydrous EtOH (200 mL), anhydrous K₂CO₃ (2.82 g, 0.020 mol, 15 mol %) is added at room temperature, followed by the tert-butyl-dimethyl-((S)-1-oxiranylmethoxy)-silane (22.2 mL, 0.11 mol, 0.78 mol %). The reaction mixture is heated to 70° C. for 6-10 h. The solvent is then removed in vacuo and the reaction mixture is taken up in EtOAc. The organic layer is washed several times with water, 0.5 N HCl, water, brine and the solvent is removed in vacuo to give the crude alcohol as a yellowish solid. The solid is suspended in diethyl ether and filtrated to give the final compound as a colorless powder. The remaining filtrate is concentrated and the process of precipitating the product with diethyl ether is repeated twice.

MS: M⁺336.3.

Melting Point: 116-118° C.

[α]²¹_{D}=−29.43 (c=0.003, MeOH).

### Example 2

### 1-[(S)-3-(tert-Butyl-dimethyl-silanyloxy)-2-(tetrahydro-pyran-2-yloxy)-propyl]-2-chloro-4-nitro-1H-imidazole (4)

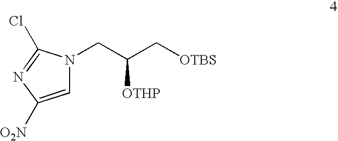

(S)-1-(tert-B utyl-dimethyl-silanyloxy)-3-(2-chloro-4-nitro-imidazol-1-yl)-propan-2-ol (3.0 g, 8.9 mmol, 100 mol %) is dissolved in dichloromethane (100 mL) and freshly distilled 3,4-dihydro-2H-pyran (1.5 g, 17.8 mmol, 200 mol %) is added to the solution, followed by pyridinium-p-toluene sulfonate (3.4 g, 13.4 mmol, 150 mol %). The reaction mixture is stirred at room temperature for 24 h. The reaction mixture is quenched with saturated aq NaHCO₃ solution. The organic layer is separated and the aqueous part is extracted with dichloromethane. The combined organic layers are washed with water, brine, dried on MgSO₄ and the solvent is removed in vacuo to give 1-[(S)-3-(tert-Butyl-dimethyl-silanyloxy)-2-(tetrahydro-pyran-2-yloxy)-propyl]-2-chloro-4-nitro-1H-imidazole as colorless oil.

MS: M⁺420.6.

### Example 3

### (S)-2-Nitro-6-(tetrahydro-pyran-2-yloxy)-6,7-dihydro-5H-imidazo[2.1-b]-[1,3]oxazine (5)

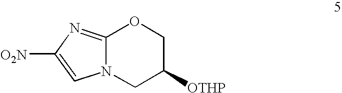

1-[(S)-3-(tert-Butyl-dimethyl-silanyloxy)-2-(tetrahydro-pyran-2-yloxy)-propyl]-2-chloro-4-nitro-1H-imidazole (0.74 g, 1.76 mmol, 100 mol %) is dissolved in anhydrous THF (180 mL) and TBAF (1 M solution in THF, 1.76 mL, 100 mol %) is added to the solution. The reaction tube is sealed and exposed to microwave at 140° C. for seven min. The solvent is removed under vacuo and the residue is purified on silica to give (S)-2-Nitro-6-(tetrahydro-pyran-2-yloxy)-6,7-dihydro-5H-imidazo[2,1-b]-[1,3]oxazine as a yellowish oil.

(S)-3-(2-Chloro-4-nitro-imidazol-1-yl)-2-(tetrahydro-pyran-2-yloxy)-propan-1-ol (0.053 g, 0.172 mmol, 100 mol %) is dissolved in anhydrous THF (17 mL) and TBAF (1 M solution in THF, 0.17 mL, 100 mol %) is added to the solution. The reaction tube is sealed and exposed to microwave at 140° C. for seven min. The solvent is removed under vacuo and the residue is purified on silica to give (S)-2-Nitro-6-(tetrahydro-pyran-2-yloxy)-6,7-dihydro-5H-imidazo[2,1-b]-[1,3]oxazine as a yellowish oil.

MS: M⁺270.

### Example 4

### (S)-2-Nitro-6,7-dihydro-5H-imidazo[2,1-b][1,3]oxazin-6-ol (6)

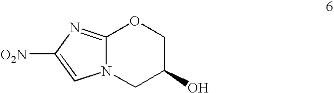

(S)-2-Nitro-6-(tetrahydro-pyran-2-yloxy)-6,7-dihydro-5H-imidazo[2,1-b]-[1,3]oxazine (4.35 g, 16.1 mmol, 100 mol %) is dissolved in HOAc/THF/Water 4:2:1 (72:36:18 ml) and the reaction mixture is heated to 60° C. and stirred for 18 h. The reaction mixture is allowed to cool to room temperature and is triturated with CH₂Cl₂ dropwise to precipitate the product. After filtration, the filtrate volume is reduced and the triturating process is repeated several times to give (S)-2-Nitro-6,7-dihydro-5H-imidazo[2,1-b][1,3]oxazin-6-ol as a yellowish solid.

MS: M⁺186.4

¹H NMR (CD₃SOCD₃, 400 MHz): δ 8.07 (s, 1H), 5.65 (s, 1H), 4.4 (dd, J=11.3, 0.9 Hz, 1H), 4.3 (dt, J=11.3, 2.4 Hz, 1H), 5.25 (m, 1H), 4.19 (dd, J=12.9, 3.3 Hz, 1H), 3.95 (dt, J=12.9, 2.4 Hz, 1H).

Melting Point: 212-214° C.

[α]²¹_{D}=−68.46 (c=0.0027, MeOH).

### Example 5

### (S)-2-Nitro-6-[2-(4-trifluoromethoxy-phenyl)-thiazol-4-yl methoxy]-6,7-dihydro-5H-imidazo[2,1-b][1,3]oxazine (II)

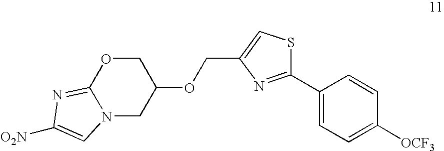

4-Trifluoromethoxy-benzamide (2.5 g, 12.2 mmol, 100%) is dissolved in 25 ml of dimethoxyethane (DME). Lawesson's reagent (2.5 g, 6.1 mmol, 50%) is added and the reaction is stirred at room temperature overnight. Reaction is concentrated and purified via silica chromatography to yield 4-trifluoromethoxy-thiobenzamide as yellow solid. (M+222.2).

4-trifluoromethoxy-thiobenzamide (3.7 g, 16.6 mmol, 100 mol %) and KHCO₃ (13.3 g, 132.7 mmol, 800 mol %) is dissolved in THF (26 mL) and sonicated for 5 min. Then, ethyl bromopyruvate (6.2 mL, 49.7 mmol, 300 mol %) is added and reaction stirred for 2 h. Reaction is cooled to 0° C. and mixture of 2,6-lutidine (16.4 mL, 141.0 mmol, 850 mol %), trifluoroacetic anhydride (9.20 mL, 66.3 mmol, 400 mol %) in THF is added. Reaction is slowly warmed to room temperature and stirred for additional 1 h. Reaction is concentrated in vacuo and EtOAc is added. Organic layer is washed two times with water, dried on MgSO₄ and the solvent is removed in vacuo. Crude material is purified using silica chromatography to yield 2-(4-trifluoromethoxy-phenyl)-thiazole-4-carboxylic acid ethyl ester as white solid (M+318.1).

2-(4-Trifluoromethoxy-phenyl)-thiazole-4-carboxylic acid ethyl ester (3.0 g, 9.5 mmol, 100 mol %) and LiAlH₄ (1.0 g, 26.7 mmol, 280 mol %) are dissolved in dry THF (20 mL) at 0° C. and reaction is stirred for 30 min. Reaction is quenched with 2 mL water followed by 1 mL of 15% NaOH solution. Solids are filtered off and washed several times with EtOAc. Filtrate is concentrated under vacuo to give [2-(4-trifluoromethoxy-phenyl)-thiazol-4-yl]-methanol.

[2-(4-trifluoromethoxy-phenyl)-thiazol-4-yl]-methanol (2.5 g, 9.1 mmol, 100 mol %) is added 33% HBR in acetic acid (access) and heated to 100° C. Reaction is cooled to 0° C. and quenched with NaOH pallets till the pH is 8.0. Product is extracted with EtOAc, dried over MgSO₄ and concentrated under vacuo to give crude oil, which is purified using silica chromatography to yield 4-bromomethyl-2-(4-trifluoromethoxy-phenyl)-thiazole.

At 0° C. under Ar, NaH (60% in mineral oil, 0.16 g, 3.9 mmol, 150 mol %) is added to stirred solution of (S)-2-Nitro-6,7-dihydro-5H-imidazo[2,1-b][1,3]oxazin-6-ol (0.49 g, 2.64 mmol, 100 mol %), 4-bromomethyl-2-(4-trifluoromethoxy-phenyl)-thiazole (1.05 g, 3.17 mmol, 120 mol %), and tetrabutylammonium iodide (0.05 g, 0.13 mmol, 5 mol %) in anhydrous DMF (10.0 mL). The mixture is allowed to warm to room temperature and stirred for overnight. The reaction is cooled to 0° C. and quenched with iced cold water. Product is extracted two times with 250 mL EtOAc, dried over MgSO₄ and concentrated under vacuo to give crude brown oil, which is purified using reverse phase preparative LC to yield (S)-2-Nitro-6-[2-(4-trifluoromethoxy-phenyl)-thiazol-4-ylmethoxy]-6,7-dihydro-5H-imidazo[2,1-b][1,3]oxazine.

MS: M⁺443.1.

¹H NMR (CD₃SOCD₃, 400 MHz): δ 8.03 (m, 3H), 7.67 (s, 1H), 7.48 (d, J=8.06 Hz, 2H), 4.78 (abq, J=12.93, 1.69 Hz, 2H), 4.70 (dt, J=11.96, 2.45 Hz, 1H), 4.48 (d, J=11.82 Hz, 1H), 4.26 (m, 3H).

Melting Point: 140-141° C.

Anal. Calcd for C₁₇H₁₃F₃N₄O₅S: C, 46.15; H, 2.97; N, 12.66. Found: C, 45.68; H, 2.71; N, 12.40.

### Example 6

### ((S)-2-Nitro-6,7-dihydro-5H-imidazo[2,1-b][1.3]oxazin-6-yloxy)-acetic acid (7)

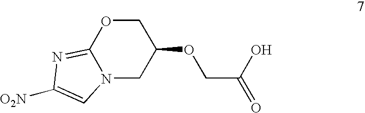

At 0° C. under Ar, NaH (60% in mineral oil, 0.13 g, 3.24 mmol, 120 mol %) is added to stirred solution of (S)-2-Nitro-6,7-dihydro-5H-imidazo[2,1-b][1,3]oxazin-6-ol (0.50 g, 2.70 mmol, 100 mol %), t-butylbromoacetate (0.48 mL, 3.20 mmol, 120 mol %), and tetrabutylammonium iodide (0.05 g, 0.14 mmol, 5 mol %) in anhydrous DMF (10.0 mL). The mixture is allowed to warm to room temperature and stirred for overnight. The reaction is cooled to 0° C. and quenched with iced cold water. Precipitate is filtered and dried under vacuo to afford ((S)-2-Nitro-6,7-dihydro-5H-imidazo[2,1-b][1,3]oxazin-6-yloxy)-acetic acid tert-butyl ester. 50% Trifluoroacetic acid in CH₂Cl₂ (100 mL) is added to the above ester (1.40 g, 4.71 mmol, 100 mol %) and allowed to stir at room temperature for 0.5 h. Solvent is removed under vacuo and the traces of TFA are removed by adding toluene followed by evaporation. This procedure is repeated till free flowing pale white solid is obtained of ((S)-2-Nitro-6,7-dihydro-5H-imidazo[2,1-b][1,3]oxazin-6-yloxy)-acetic acid.

MS: M⁻242.2.

¹H NMR (CD₃SOCD₃, 400 MHz): δ 8.04 (s, 1H), 4.59 (dt, J=111.92, 2.47 Hz, 2H), 4.43 (d, J=11.83 Hz, 2H), 4.20 (m, 3H).

Melting Point: 178-179° C.

General Procedure for Synthesis of 12 and 13

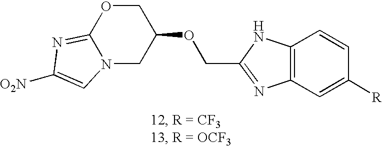

In an inert atmosphere, ((S)-2-Nitro-6,7-dihydro-5H-imidazo[2,1-b][1,3]oxazin-6-yloxy)-acetic acid (100 mol %) is dissolved in anhydrous CH₂Cl₂ (0.20 M) and added HATU (120 mol %) and DIEA (120 mol %). Reaction is stirred at room temperature for 5 min followed by addition of 1,2-diamino-substituted benzene (120 mol %). Resulting reaction mixture is stirred at room temperature overnight. Reaction is concentrated and dissolved in EtOAc and washed with water three times. Organic layer is dried under anhydrous Na₂SO₄, concentrated and purified via reverse-phase preparative LC to yield pale brown solid. This solid is dissolved in glacial acetic acid (0.33 M) and heated to 95° C. for 30 min. Crude reaction mixture is concentrated and residue obtained is purified by preparative reverse-phase LC to give white solid.

### Example 7

### (S)-2-Nitro-6-(6-trifluoromethyl-1H-benzoimidazol-2-ylmethoxy)-6,7-dihydro-5H-imidazo[2,1-b][1,3]oxazine (12)

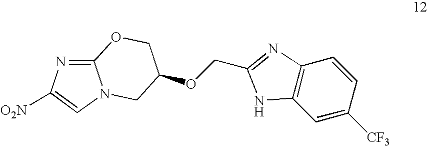

MS: M⁺383.9.

¹H NMR (CD₃SOCD₃, 400 MHz): δ 8.50 (s, 1H), 8.05 (s, 1H), 7.86 (s, 1H), 7.71 (d, J=8.43 Hz, 1H), 7.49 (dd, J=8.48, 1.45 Hz, 1H), 4.92 (s, 2H), 4.70 (dt, J=12.04, 2.50 Hz, 1H), 4.48 (d, J=11.99 Hz, 1H), 4.34 (dd, J=13.31, 1.89 Hz, 2H), 4.24 (dd, J=13.25, 2.97 Hz, 1H).

Melting Point: 120-121° C.

Anal. Calcd for C₁₉H₂₀F₃N₅O₆*HCO₂H: C, 44.76; H, 3.29; N, 16.30. Found: C, 45.26; H, 3.32; N, 16.65.

### Example 8

### (S)-2-Nitro-6-(6-trifluoromethoxy-1H-benzoimidazol-2-ylmethoxy)-6,7-dihydro-5H-imidazo[2,1-b][1,3]oxazine (13)

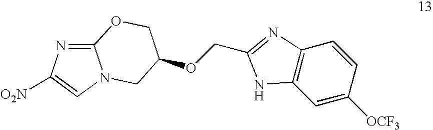

MS: M⁺399.8.

¹H NMR (CD₃SOCD₃, 400 MHz): δ 12.73 (d, J=13.72 Hz, 1H), 8.05 (s, 1H), 7.55 (m, 2H), 7.17 (m, 1H), 4.89 (s, 2H), 4.70 (dt, J=11.98, 2.39 Hz, 1H), 4.48 (d, J=11.98 Hz, 1H), 4.32 (m, 2H), 4.24 (dd, J=13.86, 3.57 Hz, 1H).

Melting Point: 99-100° C.,

[α]²¹_{D}=42.844 (c=0.0031, MeOH).

General Procedure for Synthesis of 14-21

In an inert atmosphere, ((S)-2-Nitro-6,7-dihydro-5H-imidazo[2,1-b][1,3]oxazin-6-yloxy)-acetic acid (100 mol %) is dissolved in anhydrous CH₂Cl₂ (0.2 M) and added HATU (120 mol %) and DIEA (120 mol %). Reaction is stirred at room temperature for 5 min followed by addition of amine (120 mol %). Resulting reaction mixture is stirred at room temperature overnight. Reaction is concentrated and dissolved in EtOAc and washed with water three times. Organic layer is dried under anhydrous Na₂SO₄, concentrated and purified via reverse-phase preparative LC to yield the desired product.

### Example 9

### 2-((S)-2-Nitro-6,7-dihydro-5H-imidazo[2,1-b][1,3]oxazin-6-yloxy)-N-(4-trifluoromethoxy-phenyl)-acetamide (14)

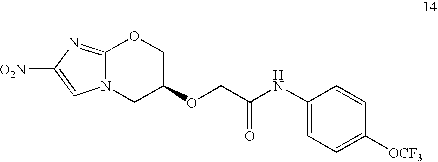

MS: M⁺403.4.

¹H NMR (CD₃SOCD₃, 400 MHz): δ 9.98 (s, 1H), 8.08 (s, 1H), 7.70 (d, J=9.09 Hz, 1H), 7.31 (d, J=8.48 Hz, 1H), 4.69 (dt, J=11.97, 2.49 Hz, 1H), 4.46 (d, J=11.86 Hz, 1H), 4.31 (m, 2H), 4.27 (s, 2H), 4.23 (dd, J=13.13, 3.02 Hz, 1H).

Melting Point: 157-158° C.

[α]²¹_{D}=−50.01 (c=0.005, MeOH).

Anal. Calcd for C₁₅H₁₃F₃N₄O₆: C, 44.78; H, 3.26; N, 13.92. Found: C, 43.67; H, 2.87; N, 13.36.

### Example 10

### 2-((S)-2-Nitro-6,7-dihydro-5H-imidazo[2,1-b][1,3]oxazin-6-yloxy)-N-(4-trifluoromethoxy-benzyl)-acetamide (15)

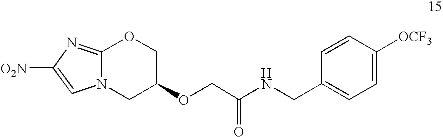

MS: M⁺417.4

¹H NMR (MeOD, 400 MHz): δ 7.73 (s, 1H), 7.35 (d, J=8.70 Hz, 2H), 7.19 (d, J=8.70 Hz, 2H), 4.72 (dt, J=12.25, 2.50 Hz, 1H), 4.45 (d, J=12.5 Hz, 1H), 4.40 (s, 2H), 4.35 (dt, J=11.7, 2.5 Hz, 1H), 4.22-4.3 (m, 2H), 4.2 (d, J=3.7 Hz, 2H).

Melting Point: 110-111° C.

[α]²¹_{D}=−46.69 (c=0.0027, MeOH).

Anal. Calcd for CO₆H₁₅F₃N₄O₆: C, 46.16; H, 3.64; N, 13.45. Found: C, 46.06; H, 3.67; N, 13.05.

### Example 11

### 2-((S)-2-Nitro-6,7-dihydro-5H-imidazo[2,1-b][1,3]oxazin-6-yloxy)-N-[4-(4-trifluoromethoxy-phenyl)-thiazol-2-yl]-acetamide (16)

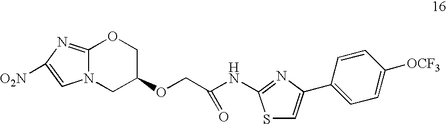

MS: M⁺486.4.

¹H NMR (CD₃SOCD₃, 400 MHz): δ 12.31 (s, 1H), 8.09 (s, 1H), 8.00 (ad, J=8.80 Hz, 2H), 7.73 (s, 1H), 7.42 (d, J=8.65 Hz, 2H), 4.68 (dt, J=11.95, 2.46 Hz, 1H), 4.46 (d, J=11.86 Hz, 1H), 4.42 (s, 2H), 4.35 (m, 2H), 4.23 (dd, J=14.09, 3.88 Hz, 1H).

Melting Point: 120-121° C.

[α]²¹_{D}=−30.67 (c=0.0028, MeOH).

Anal. Calcd for C₁₉H₂₀F₃N₅O₆*H₂O: C, 42.94; H, 3.21; N, 13.90. Found: C, 42.65; H, 3.31; N, 13.48.

### Example 12

### 2-(2-Nitro-6,7-dihydro-5H-imidazo[2,1-b][1,3]oxazin-6-yloxy)-N-(6-trifluoromethoxy-benzothiazol-2-yl)-acetamide (17)

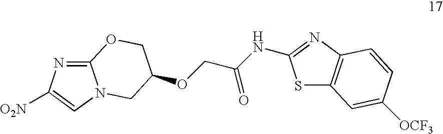

MS: M⁺460.2.

¹H NMR (CD₃SOCD₃, 400 MHz): δ 12.45 (s, 1H), 8.12 (m, 1H), 8.09 (s, 1H), 7.82 (d, J=8.82 Hz, 1H), 7.42 (m, 1H), 4.69 (dt, J=11.99, 2.53 Hz, 1H), 4.46 (d, J=11.54 Hz, 2H), 4.35 (m, 2H), 4.23 (dd, J=13.72, 3.61 Hz, 1H).

Melting Point: 89-90° C.

Anal. Calcd for C₁₆H₁₂F₃N₅O₆S*CH₃CO₂H: C, 41.62; H, 3.11; N, 13.48. Found: C, 41.53; H, 2.66; N, 13.85.

### Example 13

### 2-((S)-2-Nitro-6,7-dihydro-5H-imidazo[2,1-b][1,3]oxazin-6-yloxy)-1-[4-(4 trifluoromethoxy-phenyl)-piperazin-1-yl]-ethanone (18)

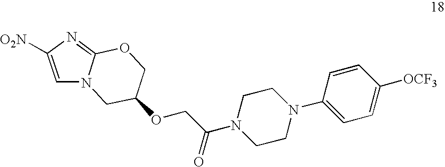

MS: M⁺472.3.

¹H NMR (MeOD, 400 MHz): δ 7.79 (s, 1H), 7.12 (d, J=9.2 Hz, 2H), 6.9 (d, J=9.2 Hz, 2H), 4.7-4.8 (m, 1H), 4.45-4.55 (m, 2H), 4.32-4.42 (m, 2H), 4.2-4.3 (m, 2H), 3.7-3.8 (m, 1H), 3.5-3.7 (m, 3H), 3.05-3.25 (m, 3H), 2.95-3.05 (m, 1H).

Melting Point: 77-79° C.

[α]²¹_{D}=−44.46 (c=0.006, MeOH).

Anal. Calcd for C₁₉H₂₀F₃N₅O₆: C, 48.41; H, 4.29; N, 18.85. Found: C, 48.28; H, 4.07; N, 14.85.

### Example 14

### 1-[4-(4-Fluoro-phenyl)-piperazin-1-yl]-2-((S)-2-nitro-6,7-dihydro-5H-imidazo[2,1-b][1,3]oxazin-6-yloxy)-ethanone (19)

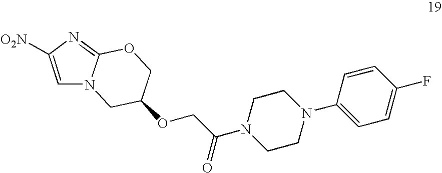

MS: M⁺406.5.

¹H NMR (CD₃SOCD₃, 400 MHz): δ 8.06 (s, 1H), 7.03 (m, 2H), 6.93 (m, 2H), 4.64 (dt, J=11.94, 2.35 Hz, 1H), 4.39 (m, 3H), 4.23 (m, 3H), 3.50 (m, 4H), 2.93 (m, 4H).

Melting Point: 73-74° C.

Anal. Calcd for C₁₈H₂₀FN₅O₅*0.5 HCO₂H: C, 51.86; H, 4.95; N, 16.34. Found: C, 51.67; H, 4.83; N, 15.68.

### Example 15

### 2-((S)-2-Nitro-6,7-dihydro-5H-imidazo[2,1-b][1,3]oxazin-6-yloxy]-1-[4-(5-trifluoromethyl-pyridin-2-yl)-piperazin-1-yl]-ethanone (20)

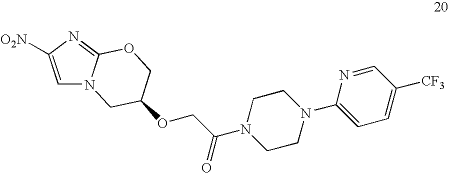

MS: M⁺475.2.

¹H NMR (CD₃SOCD₃, 400 MHz): δ 8.41 (s, 1H), 8.06 (s, 1H), 7.80 (dd, J=9.10, 2.5 Hz, 1H), 6.93 (d, J=9.09 Hz, 1H), 4.64 (dt, J=11.94, 2.35 Hz, 1H), 4.40 (m, 3H), 4.23 (m, 3H), 3.58 (m, 6H), 3.43 (m, 2H).

Melting Point: 189-190° C.

[α]²¹_{D}=−31.96 (c=0.0026, MeOH).

Anal. Calcd for C₁₈H₁₉F₃N₆O₅: C, 47.37; H, 4.20; N, 18.40. Found: C, 47.17; H, 3.77; N, 18.00.

### Example 16

### 2-((S)-2-Nitro-6,7-dihydro-5H-imidazo[2,1-b][1,3]oxazin-6-yloxy)-1-[4-(5-trifluoromethoxy-1H-benzoimidazol-2-yl)-piperidin-1-yl]-ethanone (21)

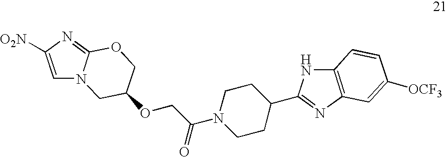

MS: M⁺511.5.

¹H NMR (CD₃SOCD₃, 400 MHz): δ 8.05 (s, 1H), 7.56 (d, J=8.66 Hz, 1H), 7.48 (s, 1H), 7.13 (d, J=8.68 Hz, 1H), 4.64 (bd, J=11.87 Hz, 1H), 4.35 (m, 6H), 3.73 (bd, J=12.03 Hz, 1H), 3.15 (m, 3H), 2.81 (t, J=11.35 Hz, 1H), 2.06 (m, 2H), 1.72 (m, 2H).

Melting Point: 131-132° C.

Anal. Calcd for C₂₁H₂₁F₃N₆O₆*H₂O*2*HCO₂H: C, 44.52; H, 4.39; N, 13.54. Found: C, 44.30; H, 3.79; N, 13.60.

### Example 17

### 4-((S)-2-nitro-6,7-dihydro-5H-imidazo[2,1-b][1,3]oxazin-6-yloxymethyl)-benzoic acid (22)

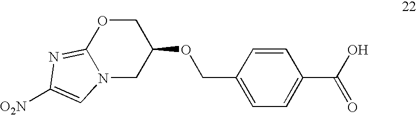

At 0° C. under Ar, NaH (60% in mineral oil, 0.26 g, 6.48 mmol, 120 mol %) is added to stirred solution of (S)-2-Nitro-6,7-dihydro-5H-imidazo[2,1-b][1,3]oxazin-6-ol (1.0 g, 5.40 mmol, 100 mol %), methyl-4-(bromomethyl)-benzoate (1.48 g, 6.48 mmol, 120 mol %), and tetrabutylammonium iodide (0.10 g, 0.27 mmol, 5 mol %) in anhydrous DMF (20.0 mL). The mixture is allowed to warm to room temperature and stirred overnight. The reaction is cooled to 0° C. and quenched with iced cold water. Precipitate is filtered and dried under vacuo to afford pale white solid.

MS: M⁺333.9.

¹H NMR (CD₃SOCD₃, 400 MHz): 658.02 (s, 1H), 7.90 (d, J=6.69 Hz, 2H), 7.43 (d, J=8.23 Hz, 2H), 4.69 (m, 3H), 4.46 (d, J=11.91 Hz, 1H), 4.25 (m, 3H), 3.83 (s, 3H).

Melting Point: 169-170° C.

1N NaOH solution (3.60 mL, 3.60 mmol, 200 mol %) is added to the above solid ester (0.60 g, 1.80 mmol, 100 mol %) in DMF (4.0 mL) and the resulting reaction mixture is heated to 95° C. overnight. Solvent is removed under vacuo followed by addition of 1.0 mL water. Reaction mixture is then acidified with glacial acetic acid to pH 2.0. Product is extracted using EtOAc (3*100 mL), combined organic layers are dried over anhydrous Na₂SO₄, and concentrated to yield 4-((S)-2-nitro-6,7-dihydro-5H-imidazo[2,1-b][1,3]oxazin-6-yloxymethyl)-benzoic acid.

MS: M⁺319.9.

¹H NMR (CD₃SOCD₃, 400 MHz): δ 12.99 (s, 1H), 8.02 (s, 1H), 7.90 (d, J=8.28 Hz, 2H), 7.41 (d, J=8.39 Hz, 2H), 4.70 (m, 3H), 4.46 (d, J=11.89 Hz, 1H), 4.24 (m, 3H).

Melting Point: 212-213° C.

### Example 18

### (S)-2-Nitro-6-[4-(5-trifluoromethoxy-1H-benzoimidazol-2-yl)-benzyloxy]-6,7-dihydro-5H-imidazo[2,1-b][1,3]oxazine (23)

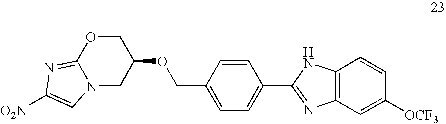

In an inert atmosphere, 4-((S)-2-nitro-6,7-dihydro-5H-imidazo[2,1-b][1,3]oxazin-6-yloxymethyl)-benzoic acid (100 mol %) is dissolved in anhydrous CH₂Cl₂ (0.20 M) and added HATU (120 mol %) and DIEA (120 mol %). Reaction is stirred at room temperature for 5 min followed by addition of 1,2-diamino-substituted benzene (120 mol %). Resulting reaction mixture is stirred at room temperature overnight. Reaction is concentrated and dissolved in EtOAc and washed with water three times. Organic layer is dried under anhydrous Na₂SO₄ and concentrated to yield pale brown solid, which is dissolved in glacial acetic acid (0.36 M). Reaction mixture is heated to 95° C. for 30 min. Crude reaction mixture is concentrated and residue obtained is purified by preparative reverse-phase LC to give final compound as fluffy solid.

MS: M⁺476.4.

¹H NMR (CD₃OH, 400 MHz): δ 8.05 (d, J=8.18 Hz, 2H), 7.74 (s, 1H), 7.62 (d, J=8.76 Hz, 1H), 7.50 (m, 3H), 7.18 (dd, J=8.73, 1.24 Hz, 1H), 4.87 (s, 2H), 4.73 (m, 2H), 4.48 (d, J=12.05 Hz, 1H), 4.30 (m, 2H).

Melting Point: 98-99° C.

[α]²¹_{D}=−37.81 (c=0.003, MeOH).

### Example 19

### 4-(2-nitro-6,7-dihydro-5H-imidazo[2,1-b][1,3]oxazin-6-yloxymethyl)-benzonitrile (24)

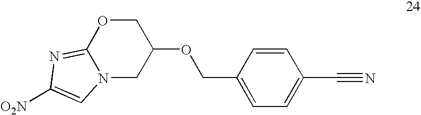

At 0° C. under Ar, NaH (60% in mineral oil, 0.16 g, 4.05 mmol, 150 mol %) is added to stirred solution of 2-Nitro-6,7-dihydro-5H-imidazo[2,1-b][1,3]oxazin-6-ol (0.50 g, 2.70 mmol, 100 mol %), 4-bromomethyl-benzonitrile (0.63 g, 3.20 mmol, 120 mol %), and tetrabutylammonium iodide (0.050 g, 0.14 mmol, 5 mol %) in anhydrous DMF (12 mL). The mixture is allowed to warm to room temperature and stirred for 4 h. The reaction is quenched with methanol, and solvent is removed under reduced pressure. Methylene chloride is added to the residue and the mixture is filtered to remove inorganic salts. Organic layer is concentrated in vacuo and purified by reverse phase LC to obtain 4-(2-nitro-6,7-dihydro-5H-imidazo[2,1-b][1,3]oxazin-6-yloxymethyl)-benzonitrile.

MS: M⁺301.2.

¹H NMR (CD₃SOCD₃, 400 MHz): δ 8.02 (s, 1H), 7.80 (d, J=8.30 Hz, 2H), 7.48 (d, J=8.34 Hz, 2H), 4.74 (abq, J=13.16, 3.32 Hz, 2H), 4.67 (dt, J=12.02, 2.37 Hz, 1H), 4.47 (d, J=11.91 Hz, 1H), 4.24 (m, 3H).

Melting Point: 166-167° C.

### Example 20

### 2-Nitro-6-[4-(5-trifluoromethoxy-benzooxazol-2-yl)-benzyloxy]-6,7-dihydro-5H-imidazo[2,1-b][1,3]oxazine (25)

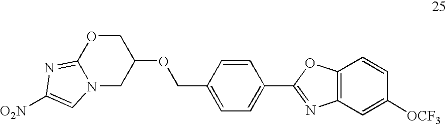

At 0° C. under Ar, acetyl chloride (3500 mol %) is added to stirred solution of 4-(2-nitro-6,7-dihydro-5H-imidazo[2,1-b][1,3]oxazin-6-yloxymethyl)-benzonitrile (100 mol %) in EtOH (0.16 M). The mixture is allowed to warm to room temperature and stirred for 5 h. The reaction is concentrated under vacuo and carried to the next step. Residue is dissolved again in dry EtOH (0.30 M), 1,2-aminophenol (120 mol %) and triethylamine (120 mol %) are added and resulting reaction mixture is stirred overnight. Reaction is concentrated and final product is purified by preparative reverse-phase LC to give final compound as brown powder.

MS: M⁺477.0.

¹H NMR (CD₃Cl, 400 MHz): δ 8.22 (d, J=8.30 Hz, 2H), 7.62 (s, 1H), 7.57 (d, J=8.82 Hz, 1H), 7.47 (d, J=8.26 Hz, 2H), 7.40 (s, 1H), 7.22 (m, 1H), 4.75 (abq, J=44.57, 12.4 Hz, 2H), 4.65 (m, 1H), 4.37 (d, J=12.16 Hz, 1H), 4.17 (m, 2H).

Melting Point: 187-188° C.

Anal. Calcd for C₂₁H₁₅F₃N₄O₆: C, 52.94; H, 3.18; N, 11.75. Found: C, 52.77; H, 3.22; N, 11.21.

### Example 21

### (S)-1-(tert-Butyl-dimethyl-silanyloxy)-3-(2-chloro-4-nitro-imidazol-1-yl)-propan-2-ol (26)

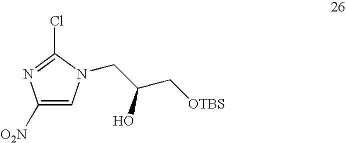

A mixture 2-chloro-4-nitroimidazole (20.0 g, 0.14 mol, 100 mol %) is dissolved in anhydrous EtOH (200 mL), anhydrous K₂CO₃ (2.829 g, 0.020 mol, 15 mol %) is added at room temperature, followed by the tert-butyl-dimethyl-((S)-1-oxiranylmethoxy)-silane (22.2 mL, 0.11 mol, 0.78 mol %). The reaction mixture is heated to 70° C. for 6-10 h. The solvent is then removed in vacuo and the reaction mixture is taken up in EtOAc. The organic layer is washed several times with water, 0.5 N HCl, water, brine and the solvent is removed in vacuo to give the crude alcohol (35.7 g, 90.2%), as a yellowish solid. The solid is suspended in diethyl ether and filtrated to give the final compound as a colorless powder. The remaining filtrate is concentrated and the process of precipitating the product with diethyl ether is repeated twice.

MS: M⁺336.3.

### Example 22

### 1-[(S)-3-(tert-Butyl-dimethyl-silanyloxy)-2-(tetrahydro-pyran-2-yloxy)-propyl]-2-chloro-4-nitro-1H-imidazole (27)

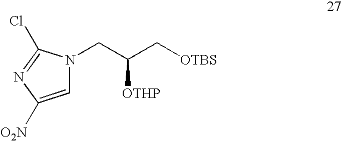

(S)-1-(tert-Butyl-dimethyl-silanyloxy)-3-(2-chloro-4-nitro-imidazol-1-yl)-propan-2-ol (3.0 g, 8.9 mmol, 100 mol %) is dissolved in dichloromethane (100 mL) and freshly distilled 3,4-dihydro-2H-pyran (1.5 g, 17.8 mmol, 200 mol %) is added to the solution, followed by pyridinium-p-toluene sulfonate (3.4 g, 13.4 mmol, 150 mol %). The reaction mixture is stirred at room temperature for 24 h. The reaction mixture is quenched with saturated aq NaHCO₃ solution. The organic layer is separated and the aqueous part is extracted with dichloromethane. The combined organic layers are washed with water, brine, dried on MgSO₄ and the solvent is removed in vacuo to give 1-[(S)-3-(tert-Butyl-dimethyl-silanyloxy)-2-(tetrahydro-pyran-2-yloxy)-propyl]-2-chloro-4-nitro-1H-imidazole as colorless oil.

MS: M⁺420.6.

### Example 23

### (S)-2-Nitro-6-(tetrahydro-pyran-2-yloxy)-6,7-dihydro-5H-imidazo[2,1-b]-[1,3]oxazine (28)

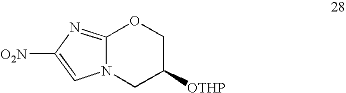

1-[(S)-3-(tert-Butyl-diethyl-silanyloxy)-2-(tetrahydro-pyran-2-yloxy)-propyl]-2-chloro-4-nitro-1H-imidazole (0.74 g, 1.76 mmol, 100 mol %) is dissolved in anhydrous THF (180 mL) and TBAF (1 M solution in THF, 1.76 mL, 100 mol %) is added to the solution. The reaction tube is sealed and exposed to microwave at 140° C. for seven min. The solvent is removed under vacuo and the residue is purified on silica to give (S)-2-Nitro-6-(tetrahydro-pyran-2-yloxy)-6,7-dihydro-5H-imidazo[2,1-b]-[1,3]oxazine as a yellowish oil.

### Example 24

### (S)-2-Nitro-6,7-dihydro-5H-imidazol[2,1-b][1,3]oxazin-6-ol (29)

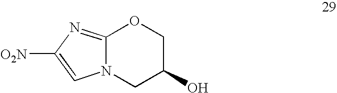

(S)-2-Nitro-6-(tetrahydro-pyran-2-yloxy)-6,7-dihydro-5H-imidazo[2,1-b]-[1,3]oxazine (4.35 g, 16.1 mmol, 100 mol %) is dissolved in HOAc/THF/Water 4:2:1 (72:36:18 ml) and the reaction mixture is heated to 60° C. and stirred for 18 h. The reaction mixture is allowed to cool to room temperature and is triturated with CH₂Cl₂ dropwise to precipitate the product. After filtration, the filtrate volume is reduced and the triturating process is repeated several times to give (S)-2-Nitro-6,7-dihydro-5H-imidazo[2,1-b][1,3]oxazin-6-ol as a yellowish solid.

MS: M⁺186.4

### Example 25

General Procedure for the Alkylation of (S)-2-Nitro-6,7-dihydro-5H-imidazo[2,1-b][1,3]oxazin-6-ol (29)

At 0° C. under Ar, NaH (60% in mineral oil, 0.16 g, 3.9 mmol, 150 mol %) is added to stirred solution of (S)-2-Nitro-6,7-dihydro-5H-imidazo[2,1-b][1,3]oxazin-6-ol (2.64 mmol, 100 mol %), benzyl halide (120 mol %), and tetrabutylammoniurn iodide (0.05 g, 0.13 mmol, 5 mol %) in anhydrous DMF (10.0 mL). The mixture is allowed to warm to room temperature and stirred for overnight. The reaction is cooled to 0° C. and quenched with iced cold water. Product is extracted two times with 250 mL EtOAc, dried over MgSO₄ and concentrated under vacuo to give crude brown oil, which is purified using reverse phase preparative LC to yield the product.

### Example 26

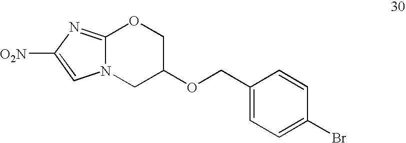

Compound 30 is prepared as described in example 25 from 2-Nitro-6,7-dihydro-5H-imidazo[2,1-b][1,3]oxazin-6-ol (6) and 1-bromo-4-chloromethylbenzene.

MS: M⁺356.3

### Example 27

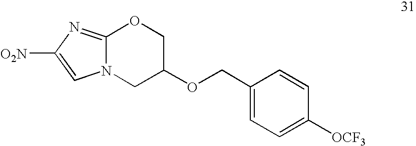

Compound 31 is prepared as described in example 25 from 2-nitro-6,7-dihydro-5H-imidazo[2,1-b][1,3]oxazin-6-ol (6) and 1-chloromethyl-4-trifluoromethoxybenzene.

MS: M⁺360.3

### Example 28

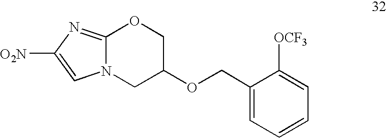

Compound 32 is prepared as described in example 25 from 2-nitro-6,7-dihydro-5H-imidazo[2,1-b][1,3]oxazin-6-ol (6) and 1-chloromethyl-2-trifluoromethoxybenzene.

MS: M⁺360.3

### Example 29

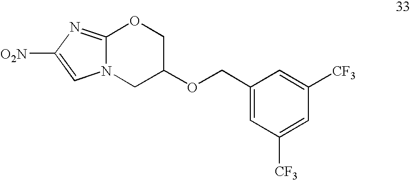

Compound 33 is prepared as described in example 25 from 2-nitro-6,7-dihydro-5H-imidazo[2,1-b][1,3]oxazin-6-ol (6) and 1-bromomethyl-3,5-ditrifluoromethylbenzene.

MS: M⁺412.3

### Example 30

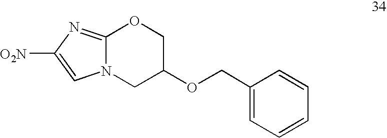

Compound 34 is prepared as described in example 25 from 2-nitro-6,7-dihydro-5H-imidazo[2,1-b][1,3]oxazin-6-ol (6) and 1-bromomethylbenzene.

MS: M⁺275.7

### Example 31

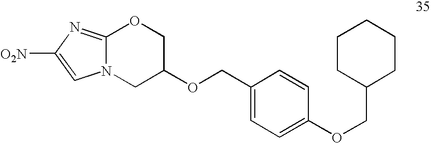

Compound 35 is prepared as described in example 25 from 2-nitro-6,7-dihydro-5H-imidazo[2,1-b][1,3]oxazin-6-ol (6) and 1-bromomethyl-4-cyclohexylmethoxybenzene.

MS: M⁺388.1

### Example 32

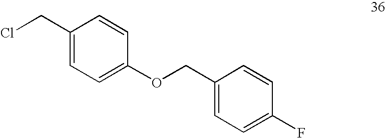

A mixture of 4-fluorobenzyl bromide (1.89 g, 10.0 mmol), 4-hydroxybenzyl alcohol (1.24 g, 10.0 mmol) and Cs₂CO₃ (6.52 g, 20.0 mmol) in anhydrous CH₃CN (30 mL) is heated under reflux for 18 h. The mixture is cooled to rt, filtered and the solvent is removed under reduced pressure to give a pale brown solid, which is triturated with small amount of CH₂Cl₂ to give 36 as a pale yellow crystalline solid.

mp: 132.0-133.4° C. ¹H NMR (CDCl₃) δ 4.63 (s, 2H), 5.03 (s, 2H), 6.93-7.11 (m, 4H).

MS: M⁺388.1

### Example 33

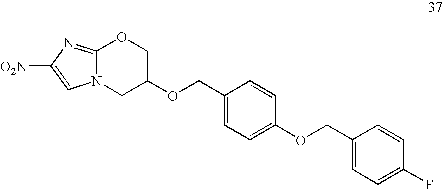

Compound 37 is prepared as described in example 25 from 2-nitro-6,7-dihydro-5H-imidazo[2,1-b][1,3]oxazin-6-ol (29) and 1-chloromethyl-4-(4-fluorobenzyloxy)benzene). Work up provided 37 as a pale yellow solid.

Mp: 150.2-150.9° C. ¹H NMR (CDCl₃) δ 4.09-4.22 (m, 3H), 4.36 (d, 12.0 Hz, 1H), 4.56-4.64 (m, 2H), 4.70 (d, 12.0 Hz, 1H), 5.06 (s, 2H), 6.96-7.15 (m, 4H), 7.25-7.46 (m, 5H). ¹³C NMR (CDCl₃) δ 48.0, 67.6, 68.9, 69.8, 70.9, 115.6, 116.0, 117.4, 130.4, 130.6, 131.1, 134.5, 148.3, 159.5, 161.7, 164.9.

### Example 34

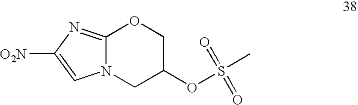

Mesyl chloride (0.78 mL, 10.0 mmol) is added to a stirred solution of 29 (0.93 g, 5.0 mmol) and triethylamine (2.1 mL, 15.0 mmol) in DMF (40 mL) at 0° C. The reaction mixture is then further stirred at 0° C. for 1 h. The solvent and excess reagents are removed under reduced pressure. H₂O (50 mL) is added to the light brown residue. The mixture is then filtered, and the solid washed with H₂O (50 mL) to give 38 as a yellow/white solid.

Mp: 213-214° C. ¹H NMR (Actone-d₆) δ 3.30 (s, 1H), 4.58 (br d, 14.1 Hz, 1H), 4.69 (dd, 3.3 Hz, 14.1 Hz, 1H), 4.77-4.78 (m, 2H), 5.56-5.60 (m, 1H), 7.86 (s, 1H). ¹³C NMR (DMSO-d₆) δ 37.9, 47.6, 68.6, 69.1, 117.9, 142.2, 146.5.

### Example 35

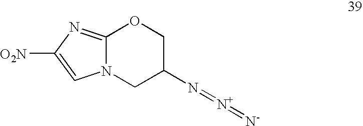

A mixture of 38 (1.68 g, 6.3 mmol), NaN₃ (5 g, 76 mmol) in DMF (20 mL) is heated at 70° C. under inert atmosphere for 48 h. The solvent is removed under reduced pressure. H₂O (100 mL) is added to the residue. The mixture is extracted with ethyl acetate (3×80 mL). The organic extracts are combined, washed with brine (200 mL), and dried over MgSO₄. The solvent is then removed under reduced pressure to give a brown solid, which is purified via column chromatography (silica gel, 0-5% methanol in methylene chloride) to give 39 as a yellow solid.

Mp: 152.0-152.4° C. ¹H NMR (acetone-d₆) δ 4.36 (dt, 2.4 Hz, 13.5 Hz, 1H), 4.58 (dd, 3.6 Hz, 13.5 Hz, 1H), 4.62-4.74 (m, 3H), 7.83 (s, 1H).

### Example 36

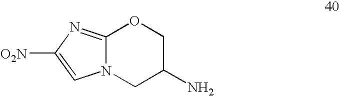

A mixture of 39 (40 mg, 0.19 mmol) and Pd/C (10% palladium on activated carbon, 38 mg) in EtOAc (8 mL) is stirred under a hydrogen atmosphere (balloon) at rt for 2 h. TLC (5% MeOH in CHCl₃) showed that the starting material is consumed. The same TLC is further developed in 25% MeOH in CHCl₃ to show one spot at R_{f}=0.20. The mixture is filtered, and the solution is concentrated under reduced pressure to give 40 as a pale brown film (17 mg, 50%). ¹H NMR (CD₃OD) δ 3.54-3.60 (m, 1H), 3.88 (ddd, 1.4 Hz, 5.5 Hz, 12.8 Hz, 1H), 4.23-4.30 (m, 2H), 4.47 (ddd, 1.6 Hz, 2.7 Hz, 12.8 Hz, 1H), 7.79 (s, 1H).

### Example 37

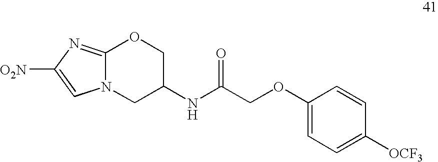

4-(Trifluoromethoxy)phenoxy acetyl chloride (1.2 g, 4.6 mmol) is added to a stirred solution of crude amine 40 (crude amine from reduction of azide, ˜1.1 mmol) and triethylamine (1.1 mL, 8.0 mmol) in DMF (20 mL) at room temperature. The reaction mixture is further stirred at rt for 18 h. The solvent and excess reagent(s) are removed under reduced pressure. H₂O (50 mL) is added. And the mixture is extracted with CH₂Cl₂ (3×50 mL). The organic extracts are combined, washed with H₂O (2×100 mL), and dried over MgSO₄. The solvent then removed under reduced pressure to give a brown gum, which is purified via prep TLC (eluted with 5% MeOH in CH₂Cl₂) to give 41 as a yellow solid.

Mp: 158.0-159.5° C. ¹H NMR (CDCl₃) δ4.18 (br d, 13.2 Hz, 1H), 4.29 (dd, 4.7 Hz, 13.2 Hz, 1H), 4.43 (dd, 1.7 Hz, 11.7 Hz, 1H), 4.53-4.62 (m, 3H), 4.78-4.88 (m, 1H), 6.82-7.14 (m, 1,4-bisubstitued pattern, 4H, aromatic H), 7.28 (s, 1H, imidazole-H), 8.20 (br d, 7.8 Hz, 1H, amide-H). Cal'd. C, 44.78; H, 3.26; N, 13.93.; analyzed C, 44.89; H, 3.25; N, 13.75.

### Example 38

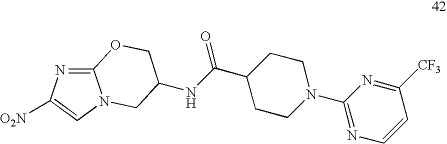

In an inert atmosphere, 40 (100 mol %) is dissolved in anhydrous DMF (0.2 M), and added 1-(4-trifluoromethyl-pyrimidin-2-yl)-piperidine-4-carbonyl chloride (120 mol %) and triethylamine (200 mol %). The resulting reaction mixture is stirred at room temperature overnight. The reaction is concentrated and dissolved in EtOAc and washed with water three times. Organic layer is dried under anhydrous Na₂SO₄, concentrated and purified via reverse-phase preparative LC to yield 42.

MS: M⁺442.3

### Example 39

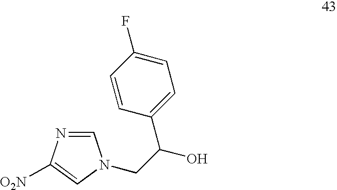

To a solution of 2-(4-fluorophenyl)-oxirane (2.17 mmol) in ethanol in a sealed tube is added 2-chloro-4-nitroimidazol *2.6 mmol) in one portion and heated at 70° C. for 16 h. The reaction mixture is concentrated and the residue is dissolved in DCM and filtered. The filtrate is concentrated to afford the crude compound. The crude compound is purified over silica gel (60-120 mesh) column using gradient of 10% to 25% ethyl acetate: pet ether as eluent to afford 204 mg of alcohol 43.

MS: M⁺251.3

### Example 40

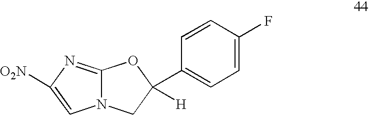

To a suspension of NaH (washed with dry hexane prior to use) (1.4 mmol) in THF is added a solution of alcohol 43 (0.7 mmol) in THF at RT and stirred at 80° C. for 2 h. The solvent is removed under vacuum and the residue is dissolved in DCM, washed with water, brine, dried (over Na₂SO₄). The organic layer is concentrated to afford crude product. The crude compound is purified over silica gel (60-120 mesh) using 15% to 25% gradient of ethyl acetate: pet ether as eluent to afford 45 mg of desired compound 44.

MS: M⁺250.2

### Example 41

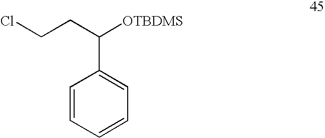

To a stirred solution of 3-chloro-1-phenyl-1-propanol (1.1 mmol) in dry DCM is added imidazole (2.33 mmol) followed by DMAP (0.11 mmol) and stirred for ½ h. Then TBDMSCI 4.68 mmol) is added and stirred for 12 h at rt and reaction is monitored by TLC. The reaction mixture diluted with water and extracted with DCM (3×30 mL) and washed with plain water, brine and dried (over with Na₂SO₄) and concentrated under vacuum below 40° C. using rotavapour.

MS: M⁺284.9

### Example 42

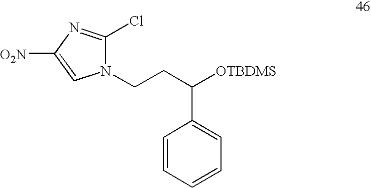

To a stirred solution of compound 45 (0.7 mmol) in dry DMF is added K₂CO₃ (1.4 mmol) and NaI (0.066 mmol) and stirred for 30 min at rt. Then compound 2-chloro-4nitro-imidazol (0.84 mmol) is added at rt and stirred for over night at 80° C. and the reaction is monitored by TLC. The reaction mixture is diluted with water and extracted with DCM (3×25 mL) and washed with water, brine and dried (over with Na₂SO₄) and concentrated under vacuum. The crude compound is purified over neutral alumina using 30% EtOAc/pet-ether as eluent to give 46.

MS: M⁺396.3

### Example 43

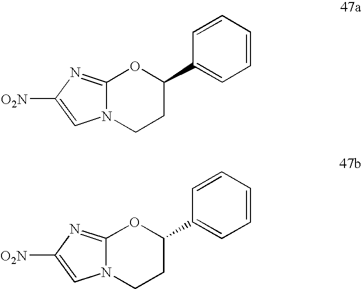

To a solution of compound 46 (0.5 mmol) in THF is added a 1 M TBAF-THF (1.5 mmol) at rt and stirred at for 12 h at 60° C. and the reaction is monitored by TLC. The reaction mixture is quenched into water and extracted with DCM (3×25 mL) and washed with water, brine and dried over with Na₂SO₄ and concentrated under vacuum at 40° C. TLC analysis of the crude compound indicates the presence of desired product and alcohol derived by the simple deprotection. These two components are separated by column chromatography over silica gel (100-200 mesh) using 40% EtOAc/pet-ether as eluent. The alcohol is again treated with TBAF-THF at 60° C. to yield an additional amount of cyclised product.

MS: M⁺246.3

### Example 44

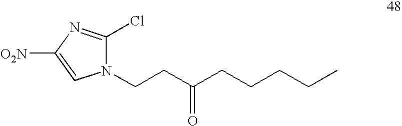

A mixture of 2-chloro-4 nitroimidazole (4.11 mmol) and triethyl amine (1.58 mmol) in 1,4-dioxane is stirred for 30 min. Then 1-octene-3-one (3.16 mmol) is added to the above mixture and stirred at 60° C. for 12 h in a sealed tube. The reaction is diluted with water and extracted into EtOAc (3×30 mL) and is washed with water, brine and dried over Na₂SO₄ and concentrated. The crude sample is used for the next step with out any further purification.

MS: M⁺274.2

### Example 45

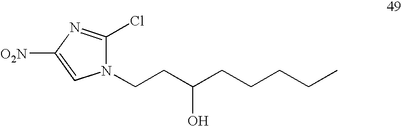

To a pre-cooled (0° C.) solution of compound 48 (2.2 mmol) in dry MeOH, is added Na₂BH₄ (2.2 mmol) portion wise and stirred for 2 h at 0° C. The reaction is quenched with acetone and stirred for 30 min followed by evaporation of the organic solvent. The reaction is diluted with water and extracted with EtOAc (3×30 mL). The organic layer is washed with water, brine and dried over Na₂SO₄ and concentrated. The crude compound is filtered through a short of column of neutral alumina using ethyl acetate as eluent. Evaporation of the solvent gave 49.

MS: M⁺276.2

### Example 46

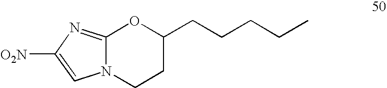

To a solution of compound 49 (1.8 mmol) in dry THF is added TBAF (5.4 mmol) slowly at room temperature and stirred for 12 h at 60° C. The reaction is diluted with water and extracted with EtOAc (3×30 mL), The organic layer is washed with water, brine and dried over Na₂SO₄ and concentrated to yield crude 50. The obtained compound is purified over silica gel (100 to 200 mesh) with 5% EtOAC: CHCl₃. Evaporation of the solvents gives pure 50.

MS: M⁺240.3

### Example 47

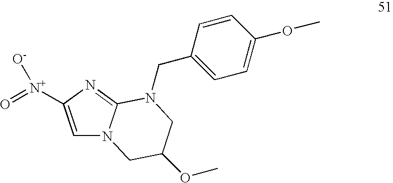

2-Azidomethyl-oxirane (6.5 g, 65.6 mmol), 2-chloro-4-nitro-1H-imidazole (10.6 g, 72.2 mmol), and potassium carbonate (1.8 g, 13.1 mmol) are mixed in dry ethanol (100 ml) and heated at 70° C. for 18 h. Solvent is removed in vacuo and the crude product is purified by flash chromatography to yield 1-azido-3-(2-chloro-4-nitro-imidazol-1-yl)-propan-2-ol as pale yellow viscous solid.

MS: 246.61; M⁺=247.2; M=245.1

¹H NMR (400 MHz, CDCl₃): δ 7.92 (s, 1H), 4.35 (bs, 1H), 4.19-4.14 (m, 2H), 4.05-3.99 (m, 1H), 3.49-3.38 (m, 2H). Sodium hydride (60% in mineral oil) and methyl iodide (120 mol %) is added to a solution of 1-azido-3-(2-chloro-4-nitro-imidazol-1-yl)-propan-2-ol (120 mol %) in anhydrous DMF (25 ml) at −20° C. and stirred for 4 h. Reaction mixture is quenched with ice cold water (100 ml) and the aqueous layer was extracted with ethyl acetate (3×100 ml). Combined organic layers are dried over anhydrous sodium sulfate, concentrated in vacuo and purified by flash chromatography to give 1-(3-azido-2-methoxy-propyl)-2-chloro-4-nitro-1H-imidazole as pale yellow viscous solid, which is reduced to the corresponding amine using Staudinger reaction. 1-(3-azido-2-methoxy-propyl)-2-chloro-4-nitro-1H-imidazole (100 mol %) and triphenylphosphine (120 mol %) are dissolved in CH₂Cl₂ and reaction stirred at rt for 6 h. Reaction is quenched using 1 N HCl to yield 3-(2-chloro-4-nitro-imidazol-1-yl)-2-methoxy-propylamine.

4-(Methoxy)benzaldehyde (150 mol %) is added to a stirred solution of 3-(2-chloro-4-nitro-imidazol-1-yl)-2-methoxy-propylamine (crude amine from reduction of azide, ˜100 mol %) in DMF at room temperature, followed by addition of glacial acetic acid (100 mol %). After 30 min, NaBH₃CN (200 mol %) is added. The reaction mixture was further stirred at rt for 18 h. The solvent is removed under reduced pressure. H₂O (50 mL) is added to the residue. The mixture is extracted with CH₂Cl₂ (3×50 mL). The organic extracts are combined, washed with H₂O (100 mL), and dried over MgSO₄. The solvent is then removed under reduced pressure to give a brown gum, which is dissolved in DMF. DBU (150 mol %) is added, and reaction irradiated in microwave for 10 min at 120° C. to yield 6-methoxy-8-(4-methoxy-benzyl)-2-nitro-5,6,7,8-tetrahydro-imidazo[1,2a]pyrimidine after preparative HPLC purification.

Mass: M⁺319.3

### Example 48

### (S)-2-Nitro-6-(4-trifluoromethylsulfanyl-benzyloxy)-6,7-dihydro-5H-imidazo[2,1-b][1,3]oxazine (52)

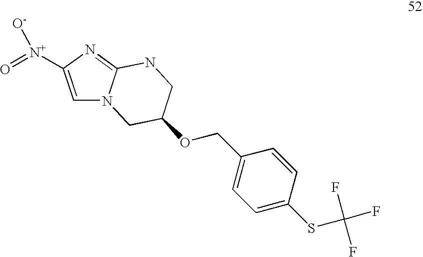

At 0° C. under Ar, NaH (60% in mineral oil, 150 mol %) is added to stirred solution of (S)-2-Nitro-6,7-dihydro-5H-imidazo[2,1-b][1,3]oxazin-6-ol (100 mol %), 1-bromomethyl-4-trifluoromethylsulfanyl-benzene (120 mol %), and tetrabutylammonium iodide (5 mol %) in anhydrous DMF. The mixture is allowed to warm to room temperature and stirred for overnight. The reaction is cooled to 0° C. and quenched with iced cold water. Product is extracted two times with 250 mL EtOAc, dried over MgSO₄ and concentrated under vacuo to give crude brown oil, which is purified using reverse phase preparative LC to yield (S)-2-Nitro-6-(4-trifluoromethylsulfanyl-benzyloxy)-6,7-dihydro-5H-imidazo[2,1-b][1,3]oxazine, 52.

Mass: M⁺376.3.

### Example 49

### 2-Nitro-6-[4-(4-trifluoromethoxy-phenyl)-piperazin-1-ylmethyl]-6,7-dihydro-5H-imidazo[2,1-b][1,3]oxazine (53)

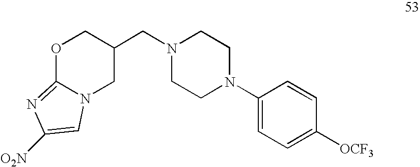

Pyridinium chlorochromate (10.54 g, 48.9 mmol) is dissolved in dichloromethane (100 ml) and celite (10 g) is added and the suspension is stirred for 30 minutes. A solution of (2,2-dimethyl-1,3-dioxan-5-yl)methanol (5 g, 34.2 mmol) in dry dichloromethane is added drop wise to the reaction mixture and stirred for 2 h at room temperature. The reaction mixture is diluted with diethyl ether (80 ml), stirred for 10 minutes, filtered through celite, washed several times with ether and the solvent is removed in vacuo to give crude 2,2-dimethyl-[1,3]dioxane-5-carbaldehyde, which is used in the next step without further purification.

2,2-dimethyl-1,3-dioxane-5-carbaldehyde (3.52 g, 24.4 mmol) is dissolved in 1,2-dichloroethane (250 ml) and 1-[4-(trifluoromethoxy)phenyl]piperazine (6.01 g, 24.4 mmol) in 1,2-dichloroethane (50 ml) is added. The reaction mixture is maintained at room temperature for 1 h. To this reaction mixture is added sodium triacetoxyborohydride (20.72 g, 97.7 mmol) in small portions. The reaction mixture is allowed to stir at room temperature for 9 h. Water is added to the reaction mixture and is extracted with chloroform. The organic layer is dried and concentrated to give 1-(2,2-dimethyl-[1,3]-dioxan-5-ylmethyl)-4-(4-trifluoromethoxy-phenyl)-piperazine.

The piperazine derivative (7.09 g, 18.9 mmol) is dissolved in methanol (80 ml), water is added (3 ml), followed by p-toluenesulfonic acid (3.91 g, 22.7 mol) and the reaction mixture is heated under reflux at 60° C. for 3 hrs. The reaction mixture is concentrated and neutralized with 10% aq. NaHCO₃ solution, followed by extraction with chloroform. The solvent is removed in vacuo to give 2-[4-(4-trifluoromethoxy-phenyl)-piperazin-1-ylmethyl]-propane-1,3-diol which is used in the next step without further purification.

A suspension of sodium hydride (2.58 g, 64.6 mmol) in dry DMF (100 ml) is cooled to −20° C. and the diol (4.5 g, 16.1 mmol) in DMF (25 ml) is added to the reaction mixture and stirred for 1 h. A solution of t-butyldimethylsilyl chloride (2.92 g, 19.3 mmol) in dry DMF is added to the reaction mixture drop wise and stirred for 1 h. The reaction mixture is quenched with ice-cold water and extracted with ethyl acetate. The organic layer is washed with water, brine, dried over Na₂SO₄ and concentrated and purified by column chromatography to give 3-(tert-butylylmethyl-silanyloxy)-2-[4-(4-trifluoromethoxy-phenyl)-piperazin-1-ylmethyl]-propan-1-ol.

¹H NMR (400 MHz, CDCl₃): δ 7.2 (d, 2H), 6.9 (d, 2H), 3.9 (dd, 1H), 3.7 (dd, 1H), 3.5 (h, 1H), 3.2 (bs, 4H), 3.2 (m, 2H), 2.8 (bs, 4H), 2.2 (m, 1H), 1.3 (s, 9H), 0.1 (s, 6H).

Mass: M⁺449.6.

The above alcohol (100 mol %) is dissolved in DCM and DMAP (0.07 mol %) is added, followed by triethylamine (1.57 mol %) and NsCl (100 mol %) at 0° C. The reaction mixture is allowed to warm to room temperature and stirred for 5 hrs. The solvent is removed in vacuo and the residue is taken up in EtOAc, washed with water, 0.5 N HCl, water, brine and dried on MgSO₄, filtered and concentrated. The residue is used in the next step without further purification.

The crude nosylate is dissolved in DMF and 2-Cl-nitroimidazole (150 mol %) and potassium carbonate (120 mol %) are added and the reaction mixture is heated in a microwave to 150° C. for 5 minutes. The solvent is removed and the residue is dissolved in EtOAc and washed several times with 0.5 N HCl, water, brine, dried on MgSO₄ and the solvent is removed in vacuo. The residue is purified by column chromatography to give 1-[3-(tert-Butyl-dimethyl-silanyloxy)-2-(2-chloro-4-nitro-imidazol-1-ylmethyl)-propyl]-4-(4-trifluoromethoxy-phenyl)-piperazine.

Mass: M⁺578.6.

1-[3-(tert-Butyl-dimethyl-si lanyloxy)-2-(2-chloro-4-nitro-imidazol-1-ylmethyl)-propyl]-4-(4-trifluoromethoxy-phenyl)-piperazine (100 mol %) is dissolved in anhydrous THF and TBAF (1 M solution, in THF, 100 mol %) is added to the solution. The reaction tube is sealed and exposed to microwave at 140° C. for eight min. The solvent is removed under vacuo and the residue is purified on silica and preparative HPLC to give compound 53 as a yellowish solid.

Mass: M⁺428.6.

### Example 50

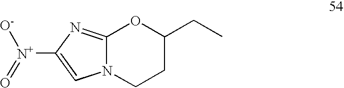

Compound 54 is synthesised as shown in the Scheme below.

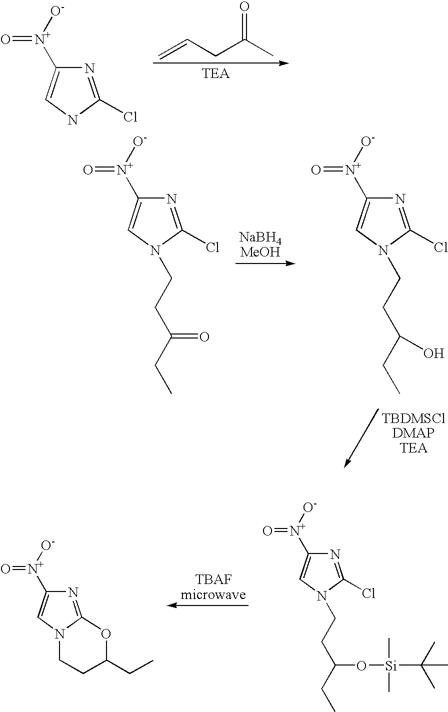

### Example 51

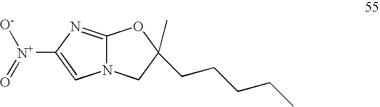

Compound 55 is synthesised as described in Eur J Med Chem 24, 1989, 631-633.

### Example 52

### Activity Against Mycobacterium Tuberculosis

The MIC values of the test and standard compounds are tested against two reference organisms Mycobacterium bovis Bacillus Calmette Guerin (BCG) Pasteur (ATCC 35745) and Mycobacterium tuberculosis H37Rv (ATCC 27294). The bacteria are cultured in Middlebrook 7H9 broth (Becton Dickinson) supplemented with 10% (v/v) albumin-dextrose saline [ADS: 0.81% NaCl, 5% BSA fraction V (Roche, Mannheim, Germany) and 2% glucose], 0.2% glycerol, 0.05% Tween-80.

The drug susceptibility testing is carried out in flat-bottom 96-well plate using the microdilution broth method (NCCLS, National Committee for Clinical Laboratory Standards. 2003 Methods for Dilution Antimicrobial Susceptibility Tests for Bacteria that Grow Aerobically; Approved Standard. Sixth Edition), with some modifications. Streptomycin and PA-824 are used as the standard drugs. Actively growing mycobacterium cultures (OD600˜0.2) are diluted in complete 7H9 broth to obtain an optical density of OD600˜0.04 (approximately 106 CFU/ml). Equal volume (100 μL) of the diluted culture is added to wells containing serially diluted drugs (100 μL). The MIC microplates are sealed to preverit evaporation and are incubated at 37° C. for 4-7 days. The growth of the bacteria is quantified either by using redox dye Alamar blue (Serotec Ltd., Oxford, UK) or by optical density (OD600) measurement.

For the Alamar blue MIC assays, 50 μl of the freshly prepared 1:1 mixture of Alamar Blue and 10% Tween-80 is added to each well. The microplates are incubated at 37° C. for another 24 hours. Bacterial growth is quantified by fluorescence measurement using excitation and emission wavelengths of 530 nm and 590 nm, respectively (SpectraMax M2, Molecular Devices) and a relative fluorescence unit (RFU) value of below 15000 is considered as no growth. Therefore, MIC is defined as the lowest drug concentration which yield a RFU reading of ≦15000.

For the turbidometry MIC method, the OD600 values of the microplates are recorded (SpectraMax M2, Molecular Devices) after 7 days. MIC is defined as the lowest drug concentration which yield an absorbance reading of ≦ 1/10 to the value obtained for the antibiotic-free growth control. Both MIC assays give consistent and reproducible results; for standard drug streptomycin, the MIC values are 06-0.13 and 0.25 μg/mL for M. bovis BCG and M. tuberculosis H37Rv, respectively.

### Example 53

### Activity Against Trypanosoma Cruzi

The Trypanosoma cruzi Tulahuen C2C4 strain is used. The infective amastigote and trypomastigote stages are cultivated in L-6 cells (a rat skeletal myoblast cell line) in RPMI 1640 medium supplemented with 2 mM L-glutamine and 10% heat-inactivated foetal bovine serum in 12.5 cm² tissue culture flasks. Amastigotes develop intracellularly, differentiate into trypomastigotes and leave the host cell. These trypomastigotes infect new L-6 cells and are the stages used to initiate an infection in the assay. All cultures and assays are conducted at 37° C. under an atmosphere of 5% CO₂ in air.

Stock compound solutions are prepared in 100% dimethylsulfoxide (DMSO) at 10 mg/mL, and heated or sonicated if necessary to dissolve the sample. After use the stocks are kept at −20° C. For the assays, the compound is further diluted to the appropriate concentration using complete medium. The DMSO concentration in the wells with the highest drug concentration does not exceed 1%.

Assays are performed in sterile 96-well microtiter plates, each well containing 100 μL medium with 2×10³ L-6 cells. After 24 hours, 50 μL of a trypanosome suspension containing 5×10³ trypomastigote bloodstream forms from culture are added to the wells. Forty-eight hours later, the medium is removed from the wells and replaced by 100 μL fresh medium, with or without a serial compound dilution. At this point the L-6 cells should be infected with amastigotes and no free trypomastigotes should be in the medium. Seven 3-fold compound dilutions are used, covering a range from 90 μg/mL to 0.123 μg/mL. Each compound is tested in duplicate. Active compounds are tested twice for confirmation. After 96 hours of incubation the plates are inspected under an inverted microscope to assure growth of the controls and sterility.

Then the substrate CPRG/Nonidet (chlorophenolred-D-galactopyranoside (CPRG, Roche Diagnostics Ltd; 15.19 mg) plus 250 μL Nonidet P40 are dissolved in 100 mL sterile phosphate-buffered saline (pH 7.2), giving 5× the final desired concentration of CPRG in 0.25% Nonidet P40/PBS), 50 μL is added to all wells. A colour reaction becomes visible within 2-6 hours and can be read photometrically at 540 nm. Data are transferred into a graphics programme, sigmoidal inhibition curves determined and IC₅₀ values calculated.

### Example 54

### Activity Against Leishmania Donovani

The Leishmania donovani strain MHOM/ET/67/L82 (obtained from Dr. S. Croft, London School of Hygiene and Tropical Medicine) is used. The strain is maintained in the Syrian Golden hamster. Amastigotes are collected from the spleen of an infected hamster. Amastigotes are grown in axenic culture at 37° C. in SM medium (Cunningham I., J. Protozool. 24, 325-329, 1977) at pH 5.4 supplemented with 10% heat-inactivated foetal bovine serum (FBS) under an atmosphere of 5% CO₂ in air.

Stock compound solutions are prepared in 100% dimethylsulfoxide (DMSO) at 10 mg/mL, and heated or sonicated if necessary to dissolve the sample. After use the stocks are kept at −20° C. For the assays, the compound is further diluted to the appropriate concentration using complete medium. The DMSO concentration in the wells with the highest drug concentration does not exceed 1%.

Assays are performed in 96-well flat-bottom microtiter plates (Costar, Corning Inc.), each well containing 100 μL of culture medium with 10⁵ amastigotes from axenic culture with or without a serial drug dilution. Concentration of amastigotes is determined in a CASY cell analysing system (Schärfe Syster™, Reutlingen, Germany). Before the amastigotes are counted, the parasite culture is passed twice through a 22 gauge needle to break up clusters of amastigotes.

The highest concentration for the test compounds is 90 μg/mL. Seven 3-fold dilutions are used, covering a range from 30 μg/mL to 0.041 μg/mL. Each compound is tested in duplicate. Active compounds are tested twice for confirmation. After 72 hours of incubation, the plates are inspected under an inverted microscope to assure growth of the controls and sterile conditions.

10 l of Alamar Blue (12.5 mg resazurin dissolved in 100 ml distilled water) are then added to each well and the plates areincubated for another 2 hours. Then the plates are read with a Spectramax Gemini XS microplate fluorometer (Molecular Devices Cooperation, Sunnyvale, Calif., USA) using an excitation wavelength of 536 nm and an emission wavelength of 588 nm.

Data are analysed using the microplate reader software Softmax Pro (Molecular Devices Cooperation, Sunnyvale, Calif., USA). Decrease of fluorescence (i.e. inhibition) is expressed as percentage of the fluorescence of control cultures and plotted against the drug concentrations. The IC₅₀ value is calculated from the sigmoidal inhibition curve by the software program.

### INDUSTRIAL APPLICABILITY

The nitroimidazole compounds of the present invention have useful pharmaceutical properties. In particular, the compounds are useful in the treatment and/or prevention of infections such as those caused by Mycobacterium tuberculosis, Trypanosoma cruzi or Leishmania donovani.

1. A compound of formula (I), or pharmaceutically acceptable salt, ester or prodrug thereof: 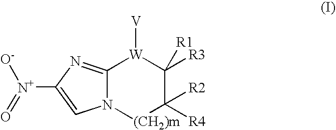
wherein:
(a) m is 0;
W is O and V is absent;
One of R1 and R3 is haloaryl and the other is H; and
R2 and R4 are both H;
or:
(b) m is 1
W is N and V is an alkylaryl group, optionally substituted with one or more alkoxy s substituents;
R1 and R3 are both H; and
One of R2 and R4 is alkoxy and the other is H;
or:
(c) m is 1;
W is O and V is absent;
one of R1 and R3 is alkyl or aryl, and the other is J; and
R2 and R4 are both H;
or:
(d) m is 1;
W is O and V is absent;
one of R2 and R4 is -L(B)ₙ-(Z)ₚ, -(L-B)_{q}-(Z)ₚ or —Y(B)_{q}-Z, and the other is H; and
R1 and R3 are both H;
wherein L is an atom group having of the formula —O—R5 where R5 is a lower alkylene, —C(O)—NH—, lower alkyenel-NH—; B is a cycloalkyl, heterocyclic, aryl or heteroaryl ring which is optionally further substituted with one or more substituents; and Z is halogen, lower alkyl substituted with at least one halogen, lower alkoxy substituted with at least one halogen or lower thioalkyl substituted with at least one halogen;
and Y is —NCH(O)—;
n is 1 or 2; p is 0, 1 or 2; and q is 1 or 2;
provided that if R2 or R4 is -L-(B)ₙ-(Z)ₚ wherein n is 1, B is phenyl and L is —O—CH₂—, then p is not 0;
and provided that if R2 or R4 is -(L-B)_{q}-(Z)ₚ wherein q is 2, both B groups are phenyl and L is —O—CH₂—, then p is not 0;
and provided that if R2 or R4 is -L-(B)ₙ-(Z)ₚ wherein n is 1, B is phenyl and L is —O—CH₂—, then Z is not 4-trifluoromethoxy, 4-fluoro, 4-trifluoroethoxy, 4-pentafluoropropoxy, 4-tetrafluoropropoxy, 4-trifluoromethyl, 2,4-difluoromethul or 2,4-difluoro.

2. A compound as claimed in claim 1, or a pharmaceutically acceptable salt, ester or prodrug there of wherein:
m is 1;
W is O and V is absent;
one of R2 and R4 is -L(B)ₙ-(Z)ₚ, -(L-B)_{q}-(Z)ₚ or —Y-(B)_{q}-Z, and the other is H; and
R1 and R3 are both H;
wherein L is an atom group having of the formula —O—R5- where R5 is a lower alkylene, —C(O)—, lower alkylene-C(O)—, —C(O)-lower alkylene, lower alkylene —C(O)—NH—, lower alkyenel-NH0; B is a cycloalkyl, heterocyclic, aryl or heteroaryl ring which is optionally further substituted with one or more substituents; and Z is halogen, lower alkyl substituted with at least one halogen, lower alkoxy substituted with at least halogen, lower alkoxy substituted with at least one halogen or lower thioalkyl substituted with at least one halogen;
and Y is —NCH(C)—;
n is 1 or 2; p is 0, 1 or 2; and q is 1 or 2;
provided that if R2 or R4 is -L-(b)ₙ-(Z)ₚ wherein n is 1, B is phenyl and L is —O—CH₂—, then p is not 0;
and provided that if R2 or R4 is -(L-B)_{q}-(Z)ₚ wherein q is 2, both B groups are phenyl and L is —O—CH₂—, the p is not 0;
and provided that if R2 or R4 is -L-(B)ₙ-Z)ₚ wherein n is 1, B is phenyl and L is —O—CH₂—, then Z is not 4-trifluoromethoxy, 4-fluoro, 4-trifluoroethoxy, 4-pentafluoropropoxy, 4-tetrafluoropropoxy, 4-trifluoromethyl, 2,4-difluoromethyl or 2,4-difluoro.

3. A compound as claimed in claim 1 or claim 2, or a pharmaceutically acceptable salt, ester or prodrug thereof, wherein one of R2 and R4 is -L-(B)ₙ-(Z)ₚ where B is a piperazine, pyridine, phenyl or benzimidazole group, or an oxazole or thiazole group, optionally fused to a phenyl ring.

4. A compound as claimed in any one of claims 1 to 3 or 2, or a pharmaceutically acceptable salt, ester or prodrug thereof, wherein L is —OCH₂C(O)—.

5. A compound of formula (II), or a pharmaceutically acceptable salt, ester or prodrug thereof: 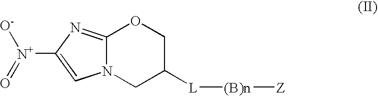
wherein:
L is an atom group having of the formula —O—R5- wherein R5 is a lower alkylene, —C(O)—, lower alkylene —C(O) lower alkylene, lower alkylene —C(O)—NH—, lower alkylene —NH—;
B is a cycloalkyl, heterocyclic, aryl or heteroaryl ring which is optionally further substituted with one or more substituents; n is 1 or 2; and Z is halogen, lower alkyl substituted with at least one halogen or lower alkoxy substituted with at least one halogen; provided that if n is 1 then B is not phenyl or if n is 1 and B is phenyl then L is —OCH₂C(O)NH— or —OCH₂C(O)NHCH₂—.

6. A pharmaceutical composition comprising a compound as claimed in any one of claims 1 2, or 5, or a pharmaceutically acceptable salt, ester or prodrug thereof, in combination with a pharmaceutically acceptable excipient, diluent or carrier.

7. A method of treating and/or preventing a disease or disorder caused by an infection by Mycobacterium tuberculosis, Trypanosoma cruzi or Leishmania donivani comprising administering to a subject in need thereof an effective amount of a compound as claimed in any one of claims 1, 2 or 5, or a pharmaceutically acceptable salt, ester or prodrug thereof.

8. A method of treating and/or preventing a disease or disorder caused by an infection by Trypanosoma cruzi or Leishmania donovani, comprising administering to a subject in need thereof an effective amount of a compound of formula (III), or a pharmaceutically acceptable salt, ester or prodrug thereof: 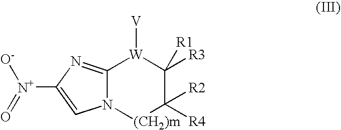
wherein:
(a) m is 0;
W is O and V is absent;
One of R1 and R3 is haloaryl or alkyl and the other is H, or R1 and R3 are both lower alkyl group; and
R2 and R4 are both H;
Or:
(b) m is 1
W is N and V is an alkylaryl group, optionally substituted with one or more alkoxy substituents;
R1 and R3 are both H; and
One of R2 and R4 is alkoxy and the other is H;
or:
(c) m is 1;
W is O and V is absent;
one of R1 and R3 is alkyl or aryl, and the other is H; and
R2 and R4 are both H;
or:
(d) m is 1;
W is O and V is absent;
one of R2 and R4 is -L(B)ₙ-(Z)ₚ, -(L-B)_{q}-(Z)ₚ or —Y-(B)_{q}-Z, and the other is H; and
R1 and R3 are both H;
wherein L is an atom group having of the formula —O—R5 where R5 is a lower alkylene, —C(O)—, —C(O)-lower alkylene, lower alkylene-C(O)—NH—, lower alkyenel-NH—; B is a cycloalkyl, heterocyclic, aryl or heteroaryl ring which is optionally further substituted with one or more substituents; and Z is halogen, lower alkyl substituted with at least one halogen, lower alkoxy substituted with at least one halogen or lower thioalkyl substituted with at least one halogen;
and Y is —NHC(O)—;
n is 1 or 2; p is 0, 1 or 2; and q is 1 or 2;
or a pharmaceutically acceptable salt, ester or prodrug thereof.

9. A method for the preparation of a nitrogen heterocyclic compound, comprising:
Reacting a non-sterically hindered substituted epoxide with a haloimidazole compound, wherein the molar ratio of the non-sterically hindered substituted epoxide to the haloimidazole compound is less than or equal to 1:1, to form an adduct with an alcohol functional group;
Protecting the alcohol functional group on the adduct to form an alcohol-protected adduct; and
treating the alcohol-protected adduct with a cyclizing agent to form the nitrogen heterocyclic compound.

10. A method as claimed in claim 9, wherein the nitrogen heterocyclic compound, in free or salt form, is represented by a compound of formula (IV) 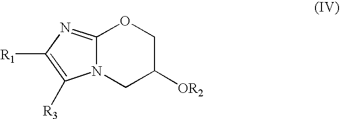
wherein R₁=nitro, acyl, formyl, sulfonyl, trifluoromethyl, cyano, halo or alkoxycarbonyl; R₂-2-tetrahydropyranyl, 2-ethoxyethyl, trityl, methyl, ethyl, allyl, trimethylsilylethoxymethyl, 2,2,2-trichloroethyl, benzyl, trimethylsilyl, t-butyldimethylsilyl, phenyldimethylsilyl, triisopropylsilyl or thexyldimethylsilyl; R₃=H, acyl, formyl, sulfonyl, trifluoromethyl, cyano, halo or alkoxycarbonyl.

11. The compound as claimed in claim 3 or a pharmaceutically acceptable salt,
ester or prodrug thereof, wherein L is —OCH₂C(0).
